(19) United States
(12) Patent Application Publication
(10) Pub. No.: US 2012/0329709 A1
(43) Pub. Date: Dec. 27, 2012

(54) PRODUCTION OF GLYCOPROTEINS

(76) Inventors: Brian Edward Collins, Arlington, MA (US); Tiffany Guo, Lagrange, OH (US); Lakshmanan Thiruneelakantapillai, Boston, MA (US); Kevin Millea, Saugus, MA (US); Dorota A. Bulik, Malden, MA (US)

(21) Appl. No.: 13/322,049

(22) PCT Filed: May 25, 2010
(86) PCT No.: PCT/US2010/036058

§ 371 Date: Aug. 24, 2012

Related U.S. Application Data
(60) Provisional application No. 61/181,103, filed on May 26, 2009.

(51) Int. Cl. C12P 21/00; A61K 38/38; A61K 38/28; A61K 38/27; A61K 38/26; A61K 38/37; C07K 14/57; C07K 14/76; C07K 14/755; C07K 16/40; C07K 14/61; C07K 16/24; C07K 14/62; G01N 27/62; C12Q 1/68; C12N 1/21; C12N 1/19; C12N 5/10; C12N 1/15; C12N 9/50; C12N 9/24; C12N 9/02; C12N 9/40; C12N 9/82; C12N 9/22; A61K 38/02

(52) U.S. Cl. 514/6.4; 514/20.9; 514/15.2; 514/11.4; 514/11.3; 514/11.7; 514/1.1; 514/14.1; 530/351; 530/362; 530/383; 530/387.3; 530/397; 530/388.15; 530/304; 435/69.1; 435/18; 435/325; 435/6.1; 435/252.33; 435/254.23; 435/254.2; 435/254.21; 435/348; 435/419; 435/412; 435/414; 435/254.11; 435/364; 435/369; 435/358; 435/354; 435/367; 435/353; 435/366; 435/370; 435/350; 435/372; 435/372.3; 435/219; 435/200; 435/189; 435/208; 435/229; 435/199

(57) ABSTRACT

The present invention provides methods and materials by which glycosylation of glycoproteins can be regulated. Methods include the monitoring and regulation of parameters such that a glycoprotein having a desired product quality is obtained.

This application claims priority to U.S. Application Ser. No. 61/181,103, filed on May 26, 2009. The disclosure of the prior application is considered part of (and is incorporated by reference in) the disclosure of this application.

### FIELD OF THE INVENTION

The invention relates to glycoproteins, the sugars thereon, and glycoprotein preparations and methods related thereto, e.g., methods of making and using the sugars, glycoproteins and glycoprotein preparations.

### BACKGROUND OF INVENTION

A typical glycoprotein includes not only an amino acid backbone but also one or more glycan moieties. The glycan complement attached to the amino acid backbone of a glycoprotein can vary structurally in many ways including, sequence, branching, sugar content, and heterogeneity. Glycosylation adds not only to the structural complexity of the molecules, but also affects or conditions many of a glycoprotein's biological and clinical attributes.

### SUMMARY OF INVENTION

The invention is based on methods of making glycoproteins. The methods can, for example, be used to produce glycoproteins having target glycan structures.

Accordingly, in one aspect, the invention features a method of inhibiting or promoting the addition of a monosaccharide moiety to an acceptor glycoprotein or protein, wherein the glycan moiety is transferred by a glycosyltransferase from a glycosyl donor to an acceptor glycoprotein or protein to provide a glycoprotein and a diphosphate nucleoside or a glycolipid, the method comprising:

providing a cell having or subject to a manipulation that increases or decreases the level of the activity of a nucleoside diphosphatase, e.g., the level of activity in the Golgi, and, e.g., which manipulation optionally promotes the formation of a target glycan structure;

culturing said cell, e.g., to provide a batch of cultured cells, e.g., to provide a glycoprotein or protein;

optionally, separating said glycoprotein or protein having a target glycan structure resulting from said inhibiting or promoting from at least one component with which said cell or batch of cells was cultured; and

optionally, analyzing said glycoprotein to confirm the presence of the target glycan structure,

thereby inhibiting or promoting the addition of a glycan moiety to an acceptor glycoprotein or protein.

In one embodiment, the method further comprises one or more of:

optionally, selecting a target glycan structure, e.g., from a list comprising a plurality of target glycan structures (in embodiments the list is also provided), and optionally memorializing said selected target glycan structure;

optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure (in embodiments the manipulation is from a list comprising a plurality of manipulations, and in embodiments the list is also provided); and

optionally, analyzing said glycoprotein to confirm the presence of the target glycan structure.

In another embodiment, the method further comprises evaluating a glycan on the surface of said cell or batch of cultured cells in order to determine if said target glycan structure is present on a glycoprotein produced by said cell or batch of cultured cells.

In another embodiment, said evaluation comprises evaluating a glycan on the surface of said cell or batch of cultured cells, to determine a property of said glycan, comparing the property to a reference, to thereby determine if said target glycan structure is present on the product.

In one embodiment, the monosaccharide is a galactosyl moiety, the glycosyl donor is UDP-galactose, the nucleoside diphosphate is UDP and the manipulation increases or decreases the level of the activity of a uridine diphosphatase, e.g., in the Golgi.

In one embodiment, the manipulation decreases the level of uridine diphosphatase activity, and the proportion of mono-galactosylated glycans is increased. In another embodiment, the level of mono-galactosylated glycans is increased in comparison with a preselected standard. In another embodiment, the amount (or proportion) of mono-galactosylated glycans is increased relative to the amount (or proportion) of mono-galactosylated glycans in a preparation of glycoproteins made by a cell or batch of cultured cells not subjected to the manipulation (in other words, a ratio of proportions).

In one embodiment, the manipulation decreases the level of uridine diphosphatase activity, and the proportion of di-galactosylated glycans is decreased. In another embodiment, the level of di-galactosylated glycans is decreased in comparison with a preselected standard. In another embodiment, the amount (or proportion) of di-galactosylated glycans is decreased relative to the amount (or proportion) of di-galactosylated glycans in a preparation of glycoproteins made by a cell or batch of cultured cells not subjected to the manipulation (in other words, a ratio of proportions).

In one embodiment, the manipulation decreases the level of uridine diphosphatase activity, and the proportion of agalactosylated glycans is increased. In another embodiment, the level of agalactosylated glycans is increased in comparison with a preselected standard. In another embodiment, the amount (or proportion) of agalactosylated glycans is increased relative to the amount (or proportion) of agalactosylated glycans in a preparation of glycoproteins made by a cell or batch of cultured cells not subjected to the manipulation (in other words, a ratio of proportions).

In one embodiment, the manipulation decreases the level of uridine diphosphatase activity, and the proportion of poly-galactosylated glycans, e.g., tri-galactosylated glycans, is decreased. In another embodiment, the manipulation decreases the level of uridine diphosphatase activity, and the proportion of poly-galactosylated glycans, e.g., tetra-galactosylated glycans, is decreased. In another embodiment, the manipulation decreases the level of uridine diphosphatase activity, and the proportion of poly-galactosylated glycans, e.g., glycans containing galαgal structures, is decreased. In another embodiment, the level of poly-galactosylated glycans is decreased in comparison with a preselected standard. In another embodiment, the amount (or proportion) of poly-galactosylated glycans is decreased relative to the amount (or proportion) of poly-galactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of mono-galactosylated glycans is decreased. In another embodiment, the level of mono-galactosylated glycans is decreased in comparison with a preselected standard. In another embodiment, the amount (or proportion) of mono-galactosylated glycans is decreased relative to the amount (or proportion) of mono-galactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of di-galactosylated glycans is increased. In another embodiment, the level of di-galactosylated glycans is increased in comparison with a preselected standard. In another embodiment, the amount (or proportion) of di-galactosylated glycans is increased relative to the amount (or proportion) of di-galactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of agalactosylated glycans is decreased. In another embodiment, the level of agalactosylated glycans is decreased in comparison with a preselected standard. In another embodiment, the amount (or proportion) of agalactosylated glycans is decreased relative to the amount (or proportion) of agalactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of poly-galactosylated glycans, e.g., tri-galactosylated glycans, is increased. In another embodiment, the manipulation increases the level of uridine diphosphatase activity, and the proportion of poly-galactosylated glycans, e.g., tetra-galactosylated glycans, is increased. In another embodiment, the manipulation increases the level of uridine diphosphatase activity, and the proportion of poly-galactosylated glycans, e.g., glycans containing galαgal structures, is increased. In another embodiment, the level of poly-galactosylated glycans is increased in comparison with a preselected standard. In another embodiment, the amount (or proportion) of poly-galactosylated glycans is increased relative to the amount (or proportion) of poly-galactosylated glycans in a preparation of glycoproteins made by a cell or batch of cultured cells not subjected to the manipulation (in other words, a ratio of proportions).

In one embodiment, the monosaccharide is a GalNAc moiety, the glycosyl donor is UDP-GalNAc, the nucleoside diphosphate is UDP and the manipulation increases or decreases the level of uridine diphosphatase activity, e.g., in the Golgi.

In one embodiment, the manipulation decreases the level of the uridine diphosphatase activity, and the site occupancy of O-glycans is decreased. In another embodiment, the site occupancy of O-glycans is decreased in comparison with a preselected standard. In another embodiment, the site occupancy of O-glycans is decreased relative to the site occupancy of O-glycans in a preparation of glycoproteins made by a cell or batch of cultured cells not subjected to the manipulation (in other words, a ratio of proportions).

In one embodiment, the manipulation increases the level of the uridine diphosphatase activity, and the site occupancy of O-glycans is increased. In another embodiment, the site occupancy of O-glycans is increased in comparison with a preselected standard. In another embodiment, the site occupancy of O-glycans is increased relative to the site occupancy of O-glycans in a preparation of glycoproteins made by a cell or batch of cultured cells not subjected to the manipulation (in other words, a ratio of proportions).

In one embodiment, the monosaccharide is a GlcNAc moiety, the glycosyl donor is UDP-GlcNAc, the nucleoside diphosphate is UDP and the manipulation increases or decreases the level of uridine diphosphatase activity, e.g., in the Golgi.

In one embodiment, the manipulation decreases the level of uridine diphosphatase activity, and the degree of branching is altered (e.g., the number of biantennary glycans is increased and the number of triantennary glycans is decreased relative to a reference).

In one embodiment, the manipulation decreases the level of uridine diphosphatase activity, and the proportion of polylactosamine or bisecting GlcNAc glycans is decreased. In another embodiment, the level of polylactosamine or bisecting GlcNAc glycans is decreased in comparison with a preselected standard. In another embodiment, the level of polylactosamine or bisecting GlcNAc glycans is decreased relative to the level of polylactosamine or bisecting GlcNAc glycans in a preparation of glycoproteins made by a cell or batch of cultured cells not subjected to the manipulation (in other words, a ratio of proportions).

In one embodiment, the manipulation increases the level of uridine diphosphatase activity, and the degree of branching is altered (e.g., the number of biantennary glycans is decreased and the number of triantennary glycans is increased relative to a reference).

In one embodiment, the manipulation increases the level of uridine diphosphatase activity, and the proportion of polylactosamine or bisecting GlcNAc glycans is increased. In another embodiment, the level of polylactosamine or bisecting GlcNAc glycans is increased in comparison with a preselected standard. In another embodiment, the level of polylactosamine or bisecting GlcNAc glycans is increased relative to the level of polylactosamine or bisecting GlcNAc glycans in a preparation of glycoproteins made by a cell or batch of cultured cells not subjected to the manipulation (in other words, a ratio of proportions).

In one embodiment, the manipulation decreases the level of uridine diphosphatase activity, and one or more of the following is present: the proportion of monogalactosylated glycans is increased, the proportion of digalactosylated glycans is decreased, the proportion of agalactosylated glycans is increased, the proportion of polygalactosylated glycans is decreased, the site occupancy of O-linked glycans is decreased, the degree of branching is altered (e.g., the number of biantennary glycans is increased and the number of triantennary glycans is decreased relative to a reference), and the level of polylactosamine or bisecting GlcNAc glycans is decreased.

In one embodiment, the manipulation increases the level of uridine diphosphatase activity, and one or more of the following is present: the proportion of monogalactosylated glycans is decreased, the proportion of digalactosylated glycans is increased, the proportion of agalactosylated glycans is decreased, the proportion of polygalactosylated glycans is increased, the site occupancy of O-linked glycans is increased, the degree of branching is altered (e.g., the number of biantennary glycans is decreased and the number of triantennary glycans is increased relative to a reference), and the level of polylactosamine or bisecting GlcNAc glycans is increased.

In one embodiment, the monosaccharide is a mannosyl moiety, the glycosyl donor is GDP-mannose, the nucleoside diphosphate is GDP and the manipulation increases or decreases the level of guanosine diphosphatase activity, e.g., in the Golgi.

In another embodiment, the manipulation decreases the level of guanosine diphosphatase activity, and the proportion of unglycosylated proteins is increased. In another embodiment, the level of unglycosylated proteins is increased in comparison with a preselected standard. In another embodiment, the level of unglycosylated proteins is increased relative to the level of unglycosylated proteins in a preparation of glycoproteins made by a cell or batch of cultured cells not subjected to the manipulation (in other words, a ratio of proportions).

In one embodiment, the manipulation decreases the level of guanosine diphosphatase activity, and the proportion of high mannose glycans is decreased. In another embodiment, the level of high mannose glycans is decreased in comparison with a preselected standard. In another embodiment, the level of high mannose glycans is decreased relative to the level of high mannose glycans in a preparation of glycoproteins made by a cell or batch of cultured cells not subjected to the manipulation (in other words, a ratio of proportions).

In one embodiment, the manipulation decreases the level of guanosine diphosphatase activity, and the proportion of complex glycans is decreased. In another embodiment, the level of complex glycans is decreased in comparison with a preselected standard. In another embodiment, the level of complex glycans is increased relative to the level of complex glycans in a preparation of glycoproteins made by a cell or batch of cultured cells not subjected to the manipulation (in other words, a ratio of proportions).

In one embodiment, the manipulation increases the level of guanosine diphosphatase activity, and the proportion of unglycosylated proteins is decreased. In another embodiment, the level of unglycosylated proteins is decreased in comparison with a preselected standard. In another embodiment, the level of unglycosylated proteins is decreased relative to the level of unglycosylated proteins in a preparation of glycoproteins made by a cell or batch of cultured cells not subjected to the manipulation (in other words, a ratio of proportions).

In one embodiment, the manipulation increases the level of guanosine diphosphatase activity, and the proportion of high mannose glycans is increased. In another embodiment, the level of high mannose glycans is increased in comparison with a preselected standard. In another embodiment, the level of high mannose glycans is increased relative to the level of high mannose glycans in a preparation of glycoproteins made by a cell or batch of cultured cells not subjected to the manipulation (in other words, a ratio of proportions).

In another embodiment, the manipulation increases the level of guanosine diphosphatase activity, and the proportion of complex glycans is increased. In another embodiment, the level of complex glycans is increased in comparison with a preselected standard. In another embodiment, the level of complex glycans is increased relative to the level of complex glycans in a preparation of glycoproteins made by a cell or batch of cultured cells not subjected to the manipulation (in other words, a ratio of proportions).

In one embodiment, the monosaccharide is a fucosyl moiety, the glycosyl donor is GDP-fucose, the nucleoside diphosphate is GDP and the manipulation increases or decreases the level of guanosine diphosphatase activity, e.g., in the Golgi.

In one embodiment, the manipulation decreases the level guanosine diphosphate activity, and the proportion of fucosylated glycans is decreased. In another embodiment, the level of fucosylated glycans is decreased in comparison with a preselected standard. In another embodiment, the level of fucosylated glycans is decreased relative to the level of afucosylated glycans in a preparation of glycoproteins made by a cell or batch of cultured cells not subjected to the manipulation (in other words, a ratio of proportions).

In one embodiment, the manipulation increases the level guanosine diphosphate activity, and the proportion of fucosylated glycans is increased. In another embodiment, the level of fucosylated glycans is increased in comparison with a preselected standard. In another embodiment, the level of fucosylated glycans is increased relative to the level of fucosylated glycans in a preparation of glycoproteins made by a cell or batch of cultured cells not subjected to the manipulation (in other words, a ratio of proportions).

In one embodiment, the manipulation decreases the level guanosine diphosphate activity, and the proportion of fucosylated glycans is decreased and the proportion of high mannose structures is decreased. In another embodiment, the manipulation increases the level guanosine diphosphate activity, and the proportion of fucosylated glycans is increased and the proportion of high mannose structures is increased.

In one embodiment, the nucleoside diphosphate is UDP, the media is further supplemented with galactose, the proportion of di-galactosylated glycans is maintained, and the degree of branching is altered (e.g., the number of biantennary glycans is increased and the number of triantennary glycans is decreased relative to a reference). In another embodiment, the nucleoside diphosphate is UDP, the media is further supplemented with glucosamine or N-acetylglucosamine, the proportion of di-galactosylated glycans is decreased, and the degree of branching is maintained.

In one embodiment, the nucleoside diphosphate is GDP, the media is further supplemented with mannose, the proportion of high-mannose glycans is maintained, and the proportion of fucosylated glycans is decreased.

In one embodiment, the glycoprotein is an N-linked glycoprotein. In another embodiment, the glycoprotein is an O-linked glycoprotein.

In one embodiment, the glycoprotein is: a cell surface receptor, e.g., CTLA4; an immunoglobulin super family member, e.g., an immunoglobulin, or portion thereof, e.g., an Fc region; a hormone, e.g., a growth factor, e.g., GCSF; an enzyme, e.g., glucocerebrosidase etc.

In one embodiment, the glycoprotein is selected from Table 1.

In one embodiment, the method further comprises isolating the glycoprotein from the cell or batch of cultured cells.

In one embodiment, the method further comprises combining the glycoprotein having a target glycan structure with a pharmaceutically acceptable component and, e.g., formulating the glycoprotein having a target glycan structure into a pharmaceutically acceptable formulation.

In one embodiment, the method further comprises evaluating (directly or indirectly) the glycan structure of the glycoprotein.

In one embodiment, evaluating comprises evaluating the level of the nucleoside diphosphate, e.g., as a proxy for the activity of the phosphatase or the presence of the target glycan structure. In another embodiment, evaluating comprises determining a value for a property of the glycan structure on the glycoprotein and comparing that value with a reference value. In another embodiment, the method further comprises memorializing the result of the evaluation.

In one embodiment, the method further comprises analyzing the glycoprotein to determine if the target glycan structure is present. In another embodiment, glycoprotein is analyzed by a method selected from the group consisting of: chromatographic methods, mass spectrometry (MS) methods, electrophoretic methods, nuclear magnetic resonance (NMR) methods, monosaccharide analysis, fluorescence methods, UV-VIS absorbance, enzymatic methods, use of a detection molecule, and combinations thereof.

In one embodiment, the method further comprises selecting one or both of a target glycan structure or a glycoprotein sequence for use in the method.

In one embodiment, the culture is supplemented with a nucleoside, e.g., uridine or guanosine. In another embodiment, the culture is supplemented with cobalt, sodium butyrate, glucosamine, ammonia, fucose, manganese, or mannose. In another embodiment, the culture is supplemented with a monosaccharide, e.g., galactose, glucosamine, N-acetylglucosamine, mannose or fucose.

In one embodiment, the manipulation is a genetic manipulation, e.g., a mutation, which decreases the level of a nucleoside diphosphatase activity, e.g., a mutation in the nucleoside diphosphatase gene. The decrease can be partial, in other words, activity is not wholly eliminated, e.g., in comparison with a gene or strain that does not have the manipulation, or complete. In an embodiment manipulation is a genetic manipulation, e.g., a mutation, which decreases, e.g., partially or completely, the level of activity of uridine diphosphatase in the product of the manipulated uridine diphosphatase gene.

In one embodiment, the nucleoside diphosphatase is uridine diphosphatase or guanosine diphosphatase.

In one embodiment, the manipulation is a genetic alteration that increases the level of a nucleoside diphosphatase activity, e.g., said cell or batch of cultured cells includes an exogenously introduced copy of a nucleoside diphosphatase gene, a duplication of a nucleoside diphosphatase gene, or a genetic alteration that places a nucleoside diphosphatase gene under control of an heterologous control element that increases the expression of the nucleoside diphosphatase gene.

In one embodiment, the manipulation is, or is the product of, a selection for a decreased nucleoside diphosphatase activity or decreased level of nucleoside diphosphatase activity.

In one embodiment, the manipulation is, or is the product of, a selection for the production of a target glycan structure, e.g., GalGlcNAc₂Man₃GlcNAc₂Fuc-Asn, a decreased glycosylation, e.g., sialylation or galactosylation, e.g., decreased mono-galactosylation or di-galactosylation, or a target decreased level of glycosylation, e.g., sialylation or galactosylation.

In one embodiment, the manipulation comprises contact with, or inclusion in or on the cell or batch of cultured cells, of an exogenous inhibitor of a nucleoside diphosphatase, e.g., a specific or non-specific inhibitor.

In one embodiment, the manipulation comprises contact with, or inclusion in or on the cell or batch of cultured cells, of a sequence specific nucleic acid-based inhibitor of the gene that encodes a nucleoside diphosphatase, e.g., an antisense nucleic acid, a siRNA, a nucleic acid aptamer, a dsRNA, a ribozyme, or a triple-helix former.

In one embodiment, the manipulation comprises contact with, or inclusion in or on the cell or batch of cultured cells, of an inhibitor of nucleoside diphosphatase activity, e.g., uridine diphosphatase activity, e.g., a phosphate mimic such as orthovanadate.

In one embodiment, the level of sialylation at one, two, three, or more preselected amino acid residues is evaluated. In another embodiment, the level of galactosylation at one, two, three, or more preselected amino acid residues is evaluated. In another embodiment, the level of fucosylation at one, two, three, or more preselected amino acid residues is evaluated. In another embodiment, the level of mannosylation at one, two, three, or more preselected amino acid residues is evaluated. In another embodiment, the level of GalNAcylation at one, two, three, or more preselected amino acid residues is evaluated.

In one embodiment, one or more of said cell or said batch of cultured cells, said manipulation, and said glycoprotein, is selected on the basis that it or the combination will provide a glycoprotein having the target glycan structure.

In one embodiment, the target glycan structure is increased, remains the same, or is decreased, as compared to what would be seen in the absence of the manipulation. Thus, although a manipulation described herein may, alter a glycan structure, e.g., decrease sialylation or galactosylation, the net result of all culture conditions may or may not alter the glycan structure.

In one embodiment, a component of the target glycan structure is transferred by a glycosyltransferase from a glycosyl donor to a protein acceptor or glycoprotein acceptor to provide said glycoprotein and a nucleoside diphosphate, and the glycosyl donor is UDP-galactose, UDP-N-acetylglucosamine, UDP-N-acetylgalactosamine, GDP-fucose or GDP-mannose.

In another aspect, the invention features a method of making, or providing, a glycoprotein having a target glycan structure, e.g., by inhibiting or promoting the addition of a monosaccharide moiety to a protein or glycoprotein wherein the monosaccharide moiety is transferred by a glycosyltransferase from a glycosyl donor to an acceptor protein or acceptor glycoprotein to provide a glycoprotein and a nucleoside diphosphate, comprising:

providing a cell having or subject to a manipulation that increases or decreases the level of the activity of a nucleoside diphosphatase, e.g., the level of activity in the Golgi, and which manipulation thereby promotes the formation of said target glycan structure;

culturing said cell, e.g., to provide a batch of cultured cells;

optionally, separating the glycoprotein or protein having a target glycan structure from at least one component with which said cell or batch of cells was cultured;

optionally, analyzing said glycoprotein to confirm the presence of the target glycan structure; and

optionally, comparing the structure of said target glycan structure present on a glycoprotein from said cultured cell or batch of cells to a reference, and determining if said target glycan structure present on a glycoprotein from said cultured cell or batch of cells differs from the corresponding glycan structure formed by a cell that lacks the manipulation

thereby providing a glycoprotein having a target glycan structure.

In one embodiment, the method further comprises one or more of:

optionally, selecting a target glycan structure, e.g., from a list comprising a plurality of target glycan structures (in embodiments the list is also provided), and optionally memorializing said selected target glycan structure;

optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure (in embodiments the manipulation is from a list comprising a plurality of manipulations, and in embodiments the list is also provided); and

optionally, analyzing said glycoprotein to confirm the presence of the target glycan structure.

In another embodiment, the method further comprises evaluating a glycan on the surface of said cell or batch of cultured cells in order to determine if said target glycan structure is present on a glycoprotein produced by said cell or batch of cultured cells.

In another embodiment, said evaluation comprises evaluating a glycan on the surface of said cell or batch of cultured cells, to determine a property of said glycan, comparing the property to a reference, to thereby determine if said target glycan structure is present on the product.

In one embodiment, the monosaccharide is a galactosyl moiety, the glycosyl donor is UDP-galactose, the nucleoside diphosphate is UDP and the manipulation increases or decreases the level of the activity of a uridine diphosphatase, e.g., in the Golgi.

In one embodiment, the manipulation decreases the level of uridine diphosphatase activity, and the proportion of mono-galactosylated glycans is increased. In another embodiment, the level of mono-galactosylated glycans is increased in comparison with a preselected standard. In another embodiment, the amount (or proportion) of mono-galactosylated glycans is increased relative to the amount (or proportion) of mono-galactosylated glycans in a preparation of glycoproteins made by a cell or batch of cultured cells not subjected to the manipulation (in other words, a ratio of proportions).

In one embodiment, the manipulation decreases the level of uridine diphosphatase activity, and the proportion of di-galactosylated glycans is decreased. In another embodiment, the level of di-galactosylated glycans is decreased in comparison with a preselected standard. In another embodiment, the amount (or proportion) of di-galactosylated glycans is decreased relative to the amount (or proportion) of di-galactosylated glycans in a preparation of glycoproteins made by a cell or batch of cultured cells not subjected to the manipulation (in other words, a ratio of proportions).

In one embodiment, the manipulation decreases the level of uridine diphosphatase activity, and the proportion of agalactosylated glycans is increased. In another embodiment, the level of agalactosylated glycans is increased in comparison with a preselected standard. In another embodiment, the amount (or proportion) of agalactosylated glycans is increased relative to the amount (or proportion) of agalactosylated glycans in a preparation of glycoproteins made by a cell or batch of cultured cells not subjected to the manipulation (in other words, a ratio of proportions).

In one embodiment, the manipulation decreases the level of uridine diphosphatase activity, and the proportion of poly-galactosylated glycans, e.g., tri-galactosylated glycans, is decreased. In another embodiment, the manipulation decreases the level of uridine diphosphatase activity, and the proportion of poly-galactosylated glycans, e.g., tetra-galactosylated glycans, is decreased. In another embodiment, the manipulation decreases the level of uridine diphosphatase activity, and the proportion of poly-galactosylated glycans, e.g., glycans containing galαgal structures, is decreased. In another embodiment, the level of poly-galactosylated glycans is decreased in comparison with a preselected standard. In another embodiment, the amount (or proportion) of poly-galactosylated glycans is decreased relative to the amount (or proportion) of poly-galactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of mono-galactosylated glycans is decreased. In another embodiment, the level of mono-galactosylated glycans is decreased in comparison with a preselected standard. In another embodiment, the amount (or proportion) of mono-galactosylated glycans is decreased relative to the amount (or proportion) of mono-galactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of di-galactosylated glycans is increased. In another embodiment, the level of di-galactosylated glycans is increased in comparison with a preselected standard. In another embodiment, the amount (or proportion) of di-galactosylated glycans is increased relative to the amount (or proportion) of di-galactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of agalactosylated glycans is decreased. In another embodiment, the level of agalactosylated glycans is decreased in comparison with a preselected standard. In another embodiment, the amount (or proportion) of agalactosylated glycans is decreased relative to the amount (or proportion) of agalactosylated glycans in a preparation of glycoproteins made by a cell or batch of cultured cells not subjected to the manipulation (in other words, a ratio of proportions).

In one embodiment, the manipulation increases the level of uridine diphosphatase activity, and the proportion of poly-galactosylated glycans, e.g., tri-galactosylated glycans, is increased. In another embodiment, the manipulation increases the level of uridine diphosphatase activity, and the proportion of poly-galactosylated glycans, e.g., tetra-galactosylated glycans, is increased. In another embodiment, the manipulation increases the level of uridine diphosphatase activity, and the proportion of poly-galactosylated glycans, e.g., glycans containing galαgal structures, is increased. In another embodiment, the level of poly-galactosylated glycans is increased in comparison with a preselected standard. In another embodiment, the amount (or proportion) of poly-galactosylated glycans is increased relative to the amount (or proportion) of poly-galactosylated glycans in a preparation of glycoproteins made by a cell or batch of cultured cells not subjected to the manipulation (in other words, a ratio of proportions).

In one embodiment, the monosaccharide is a GalNAc moiety, the glycosyl donor is UDP-GalNAc, the nucleoside diphosphate is UDP and the manipulation increases or decreases the level of uridine diphosphatase activity, e.g., in the Golgi.

In one embodiment, the manipulation decreases the level of the uridine diphosphatase activity, and the site occupancy of O-glycans is decreased. In another embodiment, the site occupancy of O-glycans is decreased in comparison with a preselected standard. In another embodiment, the site occupancy of O-glycans is decreased relative to the site occupancy of O-glycans in a preparation of glycoproteins made by a cell or batch of cultured cells not subjected to the manipulation (in other words, a ratio of proportions).

In one embodiment, the manipulation increases the level of the uridine diphosphatase activity, and the site occupancy of O-glycans is increased. In another embodiment, the site occupancy of O-glycans is increased in comparison with a preselected standard. In another embodiment, the site occupancy of O-glycans is increased relative to the site occupancy of O-glycans in a preparation of glycoproteins made by a cell or batch of cultured cells not subjected to the manipulation (in other words, a ratio of proportions).

In one embodiment, the monosaccharide is a GlcNAc moiety, the glycosyl donor is UDP-GlcNAc, the nucleoside diphosphate is UDP and the manipulation increases or decreases the level of uridine diphosphatase activity, e.g., in the Golgi.

In one embodiment, the manipulation decreases the level of uridine diphosphatase activity, and the degree of branching is altered (e.g., the number of biantennary glycans is increased and the number of triantennary glycans is decreased relative to a reference).

In one embodiment, the manipulation decreases the level of uridine diphosphatase activity, and the proportion of polylactosamine or bisecting GlcNAc glycans is decreased. In another embodiment, the level of polylactosamine or bisecting GlcNAc glycans is decreased in comparison with a preselected standard. In another embodiment, the level of polylactosamine or bisecting GlcNAc glycans is decreased relative to the level of polylactosamine or bisecting GlcNAc glycans in a preparation of glycoproteins made by a cell or batch of cultured cells not subjected to the manipulation (in other words, a ratio of proportions).

In one embodiment, the manipulation increases the level of uridine diphosphatase activity, and the degree of branching is altered (e.g., the number of biantennary glycans is decreased and the number of triantennary glycans is increased relative to a reference).

In one embodiment, the manipulation increases the level of uridine diphosphatase activity, and the proportion of polylactosamine or bisecting GlcNAc glycans is increased. In another embodiment, the level of polylactosamine or bisecting GlcNAc glycans is increased in comparison with a preselected standard. In another embodiment, the level of polylactosamine or bisecting GlcNAc glycans is increased relative to the level of polylactosamine or bisecting GlcNAc glycans in a preparation of glycoproteins made by a cell or batch of cultured cells not subjected to the manipulation (in other words, a ratio of proportions).

In one embodiment, the manipulation decreases the level of uridine diphosphatase activity, and one or more of the following is present: the proportion of monogalactosylated glycans is increased, the proportion of digalactosylated glycans is decreased, the proportion of agalactosylated glycans is increased, the proportion of polygalactosylated glycans is decreased, the site occupancy of O-linked glycans is decreased, the degree of branching is altered (e.g., the number of biantennary glycans is increased and the number of triantennary glycans is decreased relative to a reference), and the level of polylactosamine or bisecting GlcNAc glycans is decreased.

In one embodiment, the manipulation increases the level of uridine diphosphatase activity, and one or more of the following is present: the proportion of monogalactosylated glycans is decreased, the proportion of digalactosylated glycans is increased, the proportion of agalactosylated glycans is decreased, the proportion of polygalactosylated glycans is increased, the site occupancy of O-linked glycans is increased, the degree of branching is altered (e.g., the number of biantennary glycans is decreased and the number of triantennary glycans is increased relative to a reference), and the level of polylactosamine or bisecting GlcNAc glycans is increased.

In one embodiment, the monosaccharide is a mannosyl moiety, the glycosyl donor is GDP-mannose, the nucleoside diphosphate is GDP and the manipulation increases or decreases the level of guanosine diphosphatase activity, e.g., in the Golgi.

In another embodiment, the manipulation decreases the level of guanosine diphosphatase activity, and the proportion of unglycosylated proteins is increased. In another embodiment, the level of unglycosylated proteins is increased in comparison with a preselected standard. In another embodiment, the level of unglycosylated proteins is increased relative to the level of unglycosylated proteins in a preparation of glycoproteins made by a cell or batch of cultured cells not subjected to the manipulation (in other words, a ratio of proportions).

In one embodiment, the manipulation decreases the level of guanosine diphosphatase activity, and the proportion of high mannose glycans is decreased. In another embodiment, the level of high mannose glycans is decreased in comparison with a preselected standard. In another embodiment, the level of high mannose glycans is decreased relative to the level of high mannose glycans in a preparation of glycoproteins made by a cell or batch of cultured cells not subjected to the manipulation (in other words, a ratio of proportions).

In one embodiment, the manipulation decreases the level of guanosine diphosphatase activity, and the proportion of complex glycans is decreased. In another embodiment, the level of complex glycans is decreased in comparison with a preselected standard. In another embodiment, the level of complex glycans is increased relative to the level of complex glycans in a preparation of glycoproteins made by a cell or batch of cultured cells not subjected to the manipulation (in other words, a ratio of proportions).

In one embodiment, the manipulation increases the level of guanosine diphosphatase activity, and the proportion of unglycosylated proteins is decreased. In another embodiment, the level of unglycosylated proteins is decreased in comparison with a preselected standard. In another embodiment, the level of unglycosylated proteins is decreased relative to the level of unglycosylated proteins in a preparation of glycoproteins made by a cell or batch of cultured cells not subjected to the manipulation (in other words, a ratio of proportions).

In one embodiment, the manipulation increases the level of guanosine diphosphatase activity, and the proportion of high mannose glycans is increased. In another embodiment, the level of high mannose glycans is increased in comparison with a preselected standard. In another embodiment, the level of high mannose glycans is increased relative to the level of high mannose glycans in a preparation of glycoproteins made by a cell or batch of cultured cells not subjected to the manipulation (in other words, a ratio of proportions).

In another embodiment, the manipulation increases the level of guanosine diphosphatase activity, and the proportion of complex glycans is increased. In another embodiment, the level of complex glycans is increased in comparison with a preselected standard. In another embodiment, the level of complex glycans is increased relative to the level of complex glycans in a preparation of glycoproteins made by a cell or batch of cultured cells not subjected to the manipulation (in other words, a ratio of proportions).

In one embodiment, the monosaccharide is a fucosyl moiety, the glycosyl donor is GDP-fucose, the nucleoside diphosphate is GDP and the manipulation increases or decreases the level of guanosine diphosphatase activity, e.g., in the Golgi.

In one embodiment, the manipulation decreases the level guanosine diphosphate activity, and the proportion of fucosylated glycans is decreased. In another embodiment, the level of fucosylated glycans is decreased in comparison with a preselected standard. In another embodiment, the level of fucosylated glycans is decreased relative to the level of afucosylated glycans in a preparation of glycoproteins made by a cell or batch of cultured cells not subjected to the manipulation (in other words, a ratio of proportions).

In one embodiment, the manipulation increases the level guanosine diphosphate activity, and the proportion of fucosylated glycans is increased. In another embodiment, the level of fucosylated glycans is increased in comparison with a preselected standard. In another embodiment, the level of fucosylated glycans is increased relative to the level of fucosylated glycans in a preparation of glycoproteins made by a cell or batch of cultured cells not subjected to the manipulation (in other words, a ratio of proportions).

In one embodiment, the manipulation decreases the level guanosine diphosphate activity, and the proportion of fucosylated glycans is decreased and the proportion of high mannose structures is decreased. In another embodiment, the manipulation increases the level guanosine diphosphate activity, and the proportion of fucosylated glycans is increased and the proportion of high mannose structures is increased.

In one embodiment, the nucleoside diphosphate is UDP, the media is further supplemented with galactose, the proportion of di-galactosylated glycans is maintained, and the degree of branching is altered (e.g., the number of biantennary glycans is increased and the number of triantennary glycans is decreased relative to a reference). In another embodiment, the nucleoside diphosphate is UDP, the media is further supplemented with glucosamine or N-acetylglucosamine, the proportion of di-galactosylated glycans is decreased, and the degree of branching is maintained.

In one embodiment, the nucleoside diphosphate is GDP, the media is further supplemented with mannose, the proportion of high-mannose glycans is maintained, and the proportion of fucosylated glycans is decreased.

In one embodiment, the glycoprotein is an N-linked glycoprotein. In another embodiment, the glycoprotein is an O-linked glycoprotein.

In one embodiment, the glycoprotein is: a cell surface receptor, e.g., CTLA4; an immunoglobulin super family member, e.g., an immunoglobulin, or portion thereof, e.g., an Fc region; a hormone, e.g., a growth factor, e.g., GCSF; an enzyme, e.g., glucocerebrosidase etc.

In one embodiment, the glycoprotein is selected from Table 1.

In one embodiment, the method further comprises isolating the glycoprotein from the cell or batch of cultured cells.

In one embodiment, the method further comprises combining the glycoprotein having a target glycan structure with a pharmaceutically acceptable component and, e.g., formulating the glycoprotein having a target glycan structure into a pharmaceutically acceptable formulation.

In one embodiment, evaluating comprises evaluating the level of the nucleoside diphosphate, e.g., as a proxy for the activity of the phosphatase or the presence of the target glycan structure. In another embodiment, evaluating comprises determining a value for a property of the glycan structure on the glycoprotein and comparing that value with a reference value. In another embodiment, the method further comprises memorializing the result of the evaluation.

In one embodiment, the method further comprises analyzing the glycoprotein to determine if the target glycan structure is present. In another embodiment, glycoprotein is analyzed by a method selected from the group consisting of: chromatographic methods, mass spectrometry (MS) methods, electrophoretic methods, nuclear magnetic resonance (NMR) methods, monosaccharide analysis, fluorescence methods, UV-VIS absorbance, enzymatic methods, use of a detection molecule, and combinations thereof.

In one embodiment, the method further comprises selecting one or both of a target glycan structure or a glycoprotein sequence for use in the method.

In one embodiment, the culture is supplemented with a nucleoside, e.g., uridine or guanosine. In another embodiment, the culture is supplemented with cobalt, sodium butyrate, glucosamine, ammonia, fucose, manganese, or mannose. In another embodiment, the culture is supplemented with a monosaccharide, e.g., galactose, glucosamine, N-acetylglucosamine, mannose or fucose.

In one embodiment, the manipulation is a genetic manipulation, e.g., a mutation, which decreases the level of a nucleoside diphosphatase activity, e.g., a mutation in the nucleoside diphosphatase gene. The decrease can be partial, in other words, activity is not wholly eliminated, e.g., in comparison with a gene or strain that does not have the manipulation, or complete. In an embodiment manipulation is a genetic manipulation, e.g., a mutation, which decreases, e.g., partially or completely, the level of activity of uridine diphosphatase in the product of the manipulated uridine diphosphatase gene.

In one embodiment, the nucleoside diphosphatase is uridine diphosphatase or guanosine diphosphatase.

In one embodiment, the manipulation is a genetic alteration that increases the level of a nucleoside diphosphatase activity, e.g., said cell or batch of cultured cells includes an exogenously introduced copy of a nucleoside diphosphatase gene, a duplication of a nucleoside diphosphatase gene, or a genetic alteration that places a nucleoside diphosphatase gene under control of an heterologous control element that increases the expression of the nucleoside diphosphatase gene.

In one embodiment, the manipulation is, or is the product of, a selection for a decreased nucleoside diphosphatase activity or decreased level of nucleoside diphosphatase activity.

In one embodiment, the manipulation is, or is the product of, a selection for the production of a target glycan structure, e.g., GalGlcNAc₂Man₃GlcNAc₂Fuc-Asn, a decreased glycosylation, e.g., sialylation or galactosylation, e.g., decreased mono-galactosylation or di-galactosylation, or a target decreased level of glycosylation, e.g., sialylation or galactosylation.

In one embodiment, the manipulation comprises contact with, or inclusion in or on the cell or batch of cultured cells, of an exogenous inhibitor of a nucleoside diphosphatase, e.g., a specific or non-specific inhibitor.

In one embodiment, the manipulation comprises contact with, or inclusion in or on the cell or batch of cultured cells, of a sequence specific nucleic acid-based inhibitor of the gene that encodes a nucleoside diphosphatase, e.g., an antisense nucleic acid, a siRNA, a nucleic acid aptamer, a dsRNA, a ribozyme, or a triple-helix former.

In one embodiment, the manipulation comprises contact with, or inclusion in or on the cell or batch of cultured cells, of an inhibitor of nucleoside diphosphatase activity, e.g., uridine diphosphatase activity, e.g., a phosphate mimic such as orthovanadate.

In one embodiment, the level of sialylation at one, two, three, or more preselected amino acid residues is evaluated. In another embodiment, the level of galactosylation at one, two, three, or more preselected amino acid residues is evaluated. In another embodiment, the level of fucosylation at one, two, three, or more preselected amino acid residues is evaluated. In another embodiment, the level of mannosylation at one, two, three, or more preselected amino acid residues is evaluated. In another embodiment, the level of GalNAcylation at one, two, three, or more preselected amino acid residues is evaluated.

In one embodiment, one or more of said cell or said batch of cultured cells, said manipulation, and said glycoprotein, is selected on the basis that it or the combination will provide a glycoprotein having the target glycan structure.

In one embodiment, the target glycan structure is increased, remains the same, or is decreased, as compared to what would be seen in the absence of the manipulation. Thus, although a manipulation described herein may, alter a glycan structure, e.g., decrease sialylation or galactosylation, the net result of all culture conditions may or may not alter the glycan structure.

In one embodiment, a component of the target glycan structure is transferred by a glycosyltransferase from a glycosyl donor to a protein acceptor or glycoprotein acceptor to provide said glycoprotein and a nucleoside diphosphate, and the glycosyl donor is UDP-galactose, UDP-N-acetylglucosamine, UDP-N-acetylgalactosamine, GDP-fucose or GDP-mannose.

In another aspect, the invention features a method of monitoring a process, e.g., a process of culturing cells, e.g., cells of a selected type, to produce a product, comprising:

optionally, selecting a target glycan structure, e.g., from a list comprising a plurality of target glycan structures (in embodiments the list is also provided), and optionally memorializing said selected target glycan structure;

optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure (in embodiments the manipulation is from a list comprising a plurality of manipulations, and in embodiments the list is also provided);

providing a cell having or subject to a manipulation that increases or decreases the level of the activity of a nucleoside diphosphatase, e.g., the level of activity in the Golgi;

culturing said cell, e.g., to provide a batch of cultured cells; and

evaluating (directly or indirectly) a glycan complement, glycan component or glycan structure produced by the cell or the batch of cultured cells, to thereby monitor the process.

In one embodiment, the evaluating step comprises one or more of:

(a) isolating glycoproteins produced from the cell or the batch of cultured cells and evaluating the glycans containing the glycoproteins,

(b) isolating a specific glycoprotein composition produced from the cell or the batch of cultured cells and evaluating the glycans from the isolated glycoprotein composition,

(c) obtaining a glycan preparation from a glycoprotein preparation or isolated glycoprotein produced from the cell or the batch of cultured cells and evaluating the glycans in the glycan preparation,

(d) cleaving monosaccharides from glycans present on a glycoprotein produced from the cell or the batch of cultured cells or from glycans on the surface of the cell or the batch of cultured cells, and detecting the cleaved monosaccharides,

(e) providing at least one peptide from a glycoprotein preparation produced from the cell or the batch of cultured cells, and evaluating the glycans on the at least one peptide, and

(f) evaluating glycans from glycans on the cell surface of the cell or the batch of cultured cells.

In another embodiment, the evaluating step comprises isolating glycoproteins produced from the cell or the batch of cultured cells and evaluating the glycans containing the glycoproteins.

In another embodiment, the evaluating step comprises isolating a specific glycoprotein composition produced from the cell or the batch of cultured cells and evaluating the glycans from the isolated glycoprotein composition.

In another embodiment, the evaluating step comprises obtaining a glycan preparation from a glycoprotein preparation or isolated glycoprotein produced from the cell or the batch of cultured cells and evaluating the glycans in the glycan preparation.

In another embodiment, the evaluating step comprises cleaving monosaccharides from glycans present on a glycoprotein produced from the cell or the batch of cultured cells or from glycans on the surface of the cell or the batch of cultured cells, and detecting the cleaved monosaccharides.

In another embodiment, the evaluating step comprises providing at least one peptide from a glycoprotein preparation produced from the cell or the batch of cultured cells, and evaluating the glycans on the at least one peptide.

In another embodiment, the evaluating step comprises evaluating glycans from glycans on the cell surface of the cell or the batch of cultured cells.

In one embodiment, the method further comprises memorializing the result of the evaluation.

In one embodiment, evaluating comprises evaluating the level of the nucleoside diphosphate, e.g., as a proxy for the activity of the phosphatase or the presence of the target glycan structure.

In one embodiment, evaluating comprises determining a parameter related to the target glycan, e.g., the amount of a glycan structure or the ratio of a first glycan structure to a second glycan structure, on a glycoprotein produced by said cell or said batch of cultured cells, to provide an observed value for the parameter and, optionally, comparing the observed value with reference value, e.g., determining if the observed value meets a reference value for the parameter.

In one embodiment, the method further comprises, if said observed value does not meet said reference, discarding said cell or said batch of cultured cells, continuing culture of said cell or said batch of cultured cells, or altering a culture condition and further culturing said cell or said batch of cultured cells.

In one embodiment, the method further comprises, if said observed value meets said reference value, continuing culture of said cell or said batch of cultured cells, altering a culture condition and further culturing said cell or said batch of cultured cells, or discarding said cell or said batch of cultured cells.

In one embodiment, the method further comprises continuing culture of the cell or said batch of cultured cells.

In one embodiment, the method further comprises altering a culture condition and further culturing said cell or said batch of cultured cells and optionally repeating the evaluation.

In one embodiment, the parameter is the level of sialylation, the level of mono- or di-galactosylation, the ratio of mono-galactosylation:di-galactosylation, the degree of branching, the level of fucosylation, or the level of site occupancy, or other parameters disclosed herein.

In one embodiment, the glycoprotein is selected from Table 1.

In another aspect, the invention features a method of controlling a process for making a glycoprotein having a target glycan structure, comprising:

(1) providing a glycoprotein made by the process of:

optionally, selecting a target glycan structure e.g., from a list comprising a plurality of target glycan structures (in embodiments the list is also provided);

optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure (in embodiments the manipulation is from a list comprising a plurality of manipulations, and in embodiments the list is also provided);

providing a cell having or subject to a manipulation that increases or decreases the level of the activity of a nucleoside diphosphatase, e.g., the level of activity in the Golgi; and

culturing the cell to provide a glycoprotein and, e.g., to form a batch of cultured cells;

(2) evaluating (directly or indirectly) the glycan structure of the glycoprotein,

(3) responsive to said evaluation, selecting a production parameter, e.g., a culture condition, e.g., a level of a nutrient or other component in the culture medium, to thereby control the process for making a glycoprotein having a target glycan structure.

In one embodiment, the method further comprises continuing culture of the cell or batch of cultured cells under conditions that differ from those used prior to the evaluation.

In one embodiment, the method further comprises continuing culture of the cell or batch of cultured cells under the same conditions used prior to the evaluation.

In one embodiment, said evaluation step comprises comparing the structure of said target glycan structure present on a glycoprotein from said cultured cell or batch of cultured cells to a reference, and determining if said target glycan structure present on a glycoprotein from said cultured cell or batch of cultured cells differs from the corresponding glycan structure formed by a cell or batch of cultured cells that lacks the manipulation.

In another aspect, the invention features a method of making a glycoprotein, comprising:

(a) providing, acknowledging, selecting, accepting, or memorializing a defined, desired or preselected target glycan structure for the glycoprotein,

(b) optionally providing a cell manipulated to increase or decrease the level of a nucleoside diphosphatase activity,

(c) culturing a cell manipulated to increase or decrease the level of a nucleoside diphosphatase activity, e.g., to form a batch of cultured cells, and

(d) isolating from the cell or batch of cultured cells a glycoprotein having the desired target glycan structure, thereby making a glycoprotein.

In another aspect, the invention features a method of making a glycoprotein, comprising:

(a) providing, acknowledging, selecting, accepting, or memorializing a defined, desired or preselected target glycan structure for the glycoprotein, chosen, e.g., from Table 1;

(b) optionally, providing, acknowledging, selecting, accepting, or memorializing a manipulation from Table 2;

(c) culturing a cell having the manipulation, e.g., to form a batch of cultured cells;

(d) isolating from the cell or batch of cultured cells a glycoprotein having the desired target glycan structure,

thereby making a glycoprotein.

In another aspect, the invention features a method of formulating a pharmaceutical composition comprising:

contacting a glycoprotein made by a method described herein with a pharmaceutically acceptable substance, e.g., an excipient or diluent.

In another aspect, the invention features a reaction mixture comprising a manipulated cell described herein and a culture medium, optionally including secreted glycoprotein having a target glycan structure.

In another aspect, the invention features a pharmaceutical preparation of a glycoprotein described herein or made by a method described herein, wherein the glycoprotein is selected from Table 1.

In another aspect, the invention features a glycoprotein selected from Table 1 having a target glycan structure selected from Table 2.

In another aspect, the invention features a method of making, or providing, a glycoprotein having a target glycan structure, e.g., by inhibiting or promoting the addition of a monosaccharide moiety to a protein or glycoprotein wherein the monosaccharide moiety is transferred by a glycosyltransferase from a glycosyl donor to an acceptor protein or acceptor glycoprotein to provide a glycoprotein and a nucleoside diphosphate, comprising:

optionally, selecting a target glycan structure, e.g., from a list comprising a plurality of target glycan structures (in embodiments the list is provided), and optionally memorializing said selected target glycan structure;

selecting a cell, preferably on the basis that it produces a protein having the primary amino acid sequence of said glycoprotein but which protein when provided by said cell lacks said target glycan structure;

optionally, selecting a manipulation, e.g., selecting the manipulation on the basis that the manipulation increases or decreases the level of the activity of a nucleoside diphosphatase, e.g., the level of activity in the Golgi, and which manipulation thereby promotes the formation of said target glycan structure

providing said manipulation to said cell to provide a cell having or subject to a manipulation that increases or decreases the level of the activity of a nucleoside diphosphatase, e.g., the level of activity in the Golgi, and which manipulation thereby promotes the formation of said target glycan structure;

culturing said selected cell, e.g., to provide a batch of cultured cells;

optionally, separating the glycoprotein having a target glycan structure from at least one component with which the cell or said batch of cultured cells was cultured;

optionally, analyzing said glycoprotein to confirm the presence of the target glycan structure

thereby making, or providing, a glycoprotein having a target glycan structure, e.g., by inhibiting or promoting the addition of a monosaccharide moiety to a protein or glycoprotein.

In another aspect, the invention features a method of providing a cell that makes a glycoprotein having a target glycan structure, comprising:

optionally, selecting a target glycan structure, e.g., from a list comprising a plurality of target glycan structures (in embodiments the list is provided), and optionally memorializing said selected target glycan structure;

selecting a cell, preferably on the basis that it produces a protein having the primary amino acid sequence of said glycoprotein but which protein lacks said target glycan structure;

optionally, selecting a manipulation, e.g., selecting the manipulation on the basis that the manipulation increases or decreases the level of the activity of a nucleoside diphosphatase, e.g., the level of activity in the Golgi, and which manipulation thereby promotes the formation of said target glycan structure

providing said manipulation to said cell to provide a cell having or subject to a manipulation that increases or decreases the level of the activity of a nucleoside diphosphatase, e.g., the level of activity in the Golgi, and which manipulation thereby promotes the formation of said target glycan structure;

optionally producing glycoprotein from said cell and determining if said glycoprotein has said target glycan structure, thereby providing a cell that makes a glycoprotein having a target glycan structure.

In an embodiment, a cell or batch of cells expressing at least one glycoprotein is cultured under conditions so as to modulate the activity of a nucleoside diphosphatase. In some instances this may involve supplementation with a chemical inhibitor, use of a cell line with an attenuated expression of the nucleoside diphosphatase, or use of a cell line with enhanced expression of the nucleoside diphosphatase. The levels of the sugar-nucleotides may be quantified using methods known in the art so as to determine the level of inhibition. The glycoprotein product may be harvested and the product quality attributes (e.g. glycosylation) measured. In embodiments the levels of the inhibitor, transcriptional attenuation or transcriptional activation may be adjusted further so as to generate the desired product quality attributes.

In another aspect, the invention features a method of selecting a cell, e.g., a cell suitable for the production of protein having a target glycan, comprising:

optionally, selecting a target glycan structure, e.g., from a list comprising a plurality of target glycan structures (in embodiments the list is also provided), and optionally memorializing said selected target glycan structure;

optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure (in embodiments the manipulation is from a list comprising a plurality of manipulations, and in embodiments the list is also provided);

providing a cell having or subject to a manipulation that increases or decreases the level of the activity of a nucleoside diphosphatase, e.g., the level of activity in the Golgi;

culturing said cell to provide a plurality of progeny cells; and

selecting one of said progeny cells.

In an embodiment the method includes evaluating (directly or indirectly) a glycan complement, glycan component or glycan structure produced by the selected progeny cell (or progeny of the selected progeny cell).

In one embodiment, the evaluating step comprises one or more of:

(a) isolating glycoproteins produced from the selected progeny cell (or progeny of the selected progeny cell) and evaluating the glycans containing the glycoproteins,

(b) isolating a specific glycoprotein composition produced from the selected progeny cell (or progeny of the selected progeny cell) and evaluating the glycans from the isolated glycoprotein composition,

(c) obtaining a glycan preparation from a glycoprotein preparation or isolated glycoprotein produced from the selected progeny cell (or progeny of the selected progeny cell) and evaluating the glycans in the glycan preparation,

(d) cleaving monosaccharides from glycans present on a glycoprotein produced from the selected progeny cell (or progeny of the selected progeny cell) or from glycans on the surface of the selected progeny cell (or progeny of the selected progeny cell), and detecting the cleaved monosaccharides,

(e) providing at least one peptide from a glycoprotein preparation produced from the selected progeny cell (or progeny of the selected progeny cell), and evaluating the glycans on the at least one peptide, and

(f) evaluating glycans from glycans on the cell surface of the selected progeny cell (or progeny of the selected progeny cell).

In one embodiment, the method further comprises memorializing the result of the evaluation.

In one embodiment, evaluating comprises evaluating the level of the nucleoside diphosphate, e.g., as a proxy for the activity of the phosphatase or the presence of the target glycan structure.

In one embodiment, evaluating comprises determining a parameter related to the target glycan, e.g., the amount of a glycan structure or the ratio of a first glycan structure to a second glycan structure, on a glycoprotein produced by said selected progeny cell (or progeny of the selected progeny cell), to provide an observed value for the parameter and, optionally, comparing the observed value with reference value, e.g., determining if the observed value meets a reference value for the parameter.

In one embodiment, the method further comprises, if said observed value does not meet said reference, discarding said selected progeny cell (or progeny of the selected progeny cell), and repeating the process on another selected progeny cell.

In one embodiment, the glycoprotein is selected from Table 1.

Any step that generates information in a method described herein, e.g., a selection, analysis, comparison with a reference, or other evaluation or determination, can be memorialized, for example, by entry into a computer database. Such information can further be compared to a reference, or itself serve as a reference, for an evaluation made in the process.

### DETAILED DESCRIPTION

The drawings are first described.

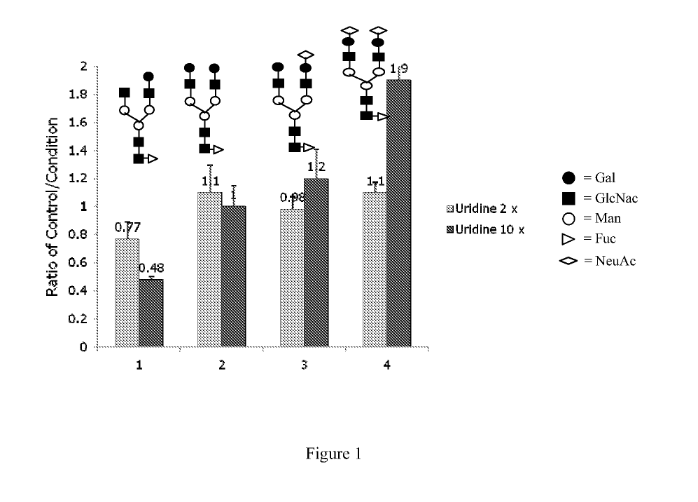

FIG. 1 is a depiction of the levels of glycans from recombinant fusion protein CTLA4-IgG produced in the presence of elevated uridine. Data are expressed as the ratio of the amount of the indicated glycan from control conditions to the amount of the same glycan produced in the presence of elevated uridine. Values are the average +/−SD of duplicate determinants. A value greater than one indicates the species is in greater abundance under the control conditions.

### DEFINITIONS

“Branched glycan” as used herein refers to a glycan in which one monosaccharide is involved in more than two glycosidic linkages and serves as a branchpoint. Branched glycans may be, e.g., di-, tri- or tetra-antennary.

“Culturing” as used herein refers to placing a cell under conditions that allow for at least some of the steps for the production of a glycoprotein to proceed. In embodiments the conditions are sufficient to allow the glycosylation process to be completed. In embodiments the conditions are sufficient to allow all of the steps, e.g., through secretion, to occur. Culturing refers to cultures of cells, cell lines, and populations of cells. The cells can be eukaryotic or a prokaryotic cells, e.g., animal, plant, yeast, fungal, insect or bacterial cells. In embodiments culturing refers to in vitro culture of cells, e.g., primary or secondary cell lines. In an embodiment the cell is, e.g., a vertebrate, mammalian or rodent cell. Culturing does not include production by a cell in an animal, e.g., a transgenic animal such as a transgenic mouse or goat, or in a plant, e.g., a transgenic plant.

“Degree of branching” as used herein refers to the relative extent of branching in a glycan. For example, a glycan with a high degree of branching may have a higher number of triantennary glycans and a lower number of biantennary glycans at a selected position.

“Glycan complement” as used herein refers to all of the glycan components of a glycoprotein or glycolipid. In the case of a protein having a single glycosylation site, the glycan component attached thereto forms the glycan complement. In the case of a protein having more than one glycosylation site, the glycan complement is made up of the glycan components attached at all of the sites. The N-linked glycan complement refers to all of the N-linked glycan components of a protein. The O-linked glycan complement refers to all of the O-linked glycan components of a protein. A “component of the glycan complement” refers to a subset of the glycan components making up the glycan complement, e.g., one or more glycan components attached to its or their respective glycosylation site or sites.

“Glycan component” as used herein refers to a sugar moiety, e.g., a monosaccharide, oligosaccharide or polysaccharide (e.g., a disaccharide, trisaccharide, tetrasaccharide, etc.) attached to a protein at one site. In embodiments the attachment is covalent and the glycan component is N- or O-linked to the protein. Glycan components can be chains of monosaccharides attached to one another via glycosidic linkages. Glycan components can be linear or branched.

“Glycan structure” as used herein refers to the structure of a glycan complement, component of a glycan complement, or glycan component. Elements of glycan structure include one or more of the following:

the presence, absence, or level of glycosylation at one or more sites, e.g., one or more sites for N-linked or O-linked glycosylation;

N- or O-linkage;

length (number of monosaccharide moieties);

placement or position of a monosaccharide, e.g., a galactosyl moiety, within a chain;

saccharide content (e.g., the amounts or ratios of the monosaccharide components in a particular glycan);

saccharide sequence (e.g., the order of monosaccharide subunits in a glycan moiety);

the presence, absence or amount of a terminal or penultimate saccharide subunit;

the number, placement, and type (e.g., the presence, absence or amount of bisecting GlcNAc or mannose structures) of branch points;

the presence, absence or level of a complex structure, e.g., biantennary structure, triantennary structure, tetraantennary structure, etc.

the presence, absence or level of a high mannose or a hybrid structure;

the relationship between monosaccharide moieties (e.g., linkages between monosaccharide moieties, isomers and branch points);

the presence, absence, position, or number of a selected monosaccharide, e.g., a galactosyl moiety, fucosyl moiety, GlcNAc moiety, or mannosyl moiety.

the presence, absence, position or number of a selected structure, e.g., a mono-galactosylated, digalactosylated, or polygalactosylated structure. Other nonlimiting examples include any other structure found on naturally occurring glycoproteins; and

heterogeneity or homogeneity across one or more sites (e.g., diversity across the entire protein, e.g., the degree of occupancy of potential glycosylation sites of a protein (e.g., the degree of occupancy of the same potential glycosylation site between two or more of the particular protein backbones in a plurality of molecules and the degree of occupancy of one potential glycosylation site on a protein backbone relative to a different potential glycosylation site on the same protein backbone).

A glycan structure can be described in terms of a comparison of the presence, absence or amount of a first glycan structure to a second glycan structure. For example, the presence, absence or amount of sialic acid relative to the presence, absence or amount of fucose. In other examples, the presence, absence or amount of a sialic acid such as N-acetylneuraminic acid can be compared, e.g., to the presence, absence or amount of a sialic acid derivative such as N-glycolylneuraminic acid.

Glycan structures can be described, identified or assayed in a number of ways. A glycan structure can be described, e.g., in defined structural terms, e.g., by chemical name, or by a functional or physical property, e.g., by molecular weight or by a parameter related to purification or separation, e.g., retention time of a peak in a column or other separation device. In embodiments a glycan structure can, by way of example, be a peak or other fraction (representing one or more species) from glycan structures derived from a glycoprotein, e.g., from an enzymatic digest.

“Manipulation” as used herein can be any of a nucleoside diphosphate activity (NDPA) manipulation, an envirocultural manipulation, or a selected functional manipulation. In general a manipulation is induced, selected, isolated, engineered, or is otherwise the product of the “hand of man.”

A “nucleoside diphosphate activity (NDPA) manipulation” as used herein refers to a property of a cell that increases or decreases the level of nucleoside diphosphatase activity, e.g., in the Golgi. Increased or decreased means by comparison with a cell that is not subject to the NDPA manipulation.

Examples of NDPA manipulations include:

the presence in or on the cell of an exogenous inhibitor (e.g., an siRNA or a phosphatase inhibitor) or enhancer of the level of nucleoside diphosphatase activity; or

a mutation or other genetic event that inhibits or enhances the level of nucleoside diphosphatase activity.

An “envirocultural manipulation” as used herein refers to a property of the culture conditions, e.g., of the culture medium, that alters the abundance of the nucleoside diphosphate in relation to the nucleoside diphosphatase activity, and results in a decrease or increase in transfer of a monosaccharide moiety to a protein or glycoprotein. Examples include the addition of a nucleoside, e.g., uridine or guanosine, a component that alters the level the level of a nucleoside diphosphate, or a component that alters the subcellular concentration of the nucleoside diphosphate, e.g., in the Golgi. Examples of media conditions that will lead to altered concentrations of UDP sugars include but are not limited to altering the levels of cobalt, sodium butyrate, glucosamine, ammonia, manganese, mannose, and uridine. Examples of media conditions that will lead to altered levels of GDP sugars include but are not limited to altering the levels of cobalt, butyrate, fucose, guanosine, and manganese.

A selected functional manipulation is a physical characteristic or property characterized, e.g., by the process that gave rise to it, e.g., a cell that was placed under selective conditions that result in the cell being able to produce a glycoprotein having a target glycan structure, wherein the underlying basis for the ability to produce said glycoprotein having a target glycan structure may or may not be known or characterized.

“Monosaccharide” as used herein refers to the basic unit of a glycan component and in embodiments, a moiety that is transferred by a glycosyltransferase onto a substrate. Monosaccharides, as used herein, include naturally and non-naturally occurring monosaccharides. Exemplary monosaccharide moieties include glucose (Glc), N-acetylglucosamine (GlcNAc), mannose (Man), N-acetylmannosamine (ManNAc), galactose (Gal), N-acetylgalactosamine (GalNAc), fucose (Fuc), sialic acid (NeuAc) and ribose, as well as derivatives and analogs thereof. Derivatives of various monosaccharides are known. For example, sialic acid encompasses over thirty derivatives with N-acetylneuraminic acid and N-glycolylneuraminic acid forming the core structures. Examples of sialic acid analogs include those that functionally mimic sialic acid, but are not recognized by endogenous host cell sialylases. Other examples of monosaccharide analogs include, but are not limited to, N-levulinoylmannosamine (ManLev), Neu5Acα-methyl glycoside, Neu5Acβ-methyl glycoside, Neu5Acα-benzyl glycoside, Neu5Acβ-benzyl glycoside, Neu5Acα-methylglycoside methyl ester, Neu5Acα-methyl ester, 9-O-Acetyl-N-acetylneuraminic acid, 9-O-Lactyl-N-acetylneuraminic acid, N-azidoacetylmannosamine and O-acetylated variations thereof, and Neu5Acα-ethyl thioglycoside.

A “target glycan structure” as used herein refers to a glycan complement (or component of a glycan complement), having a selected or specified glycan structure. An example of a selected or specific glycan structure is one that has a selected or specific value for the amount of mono-, di- or polygalactosylation, amount of branching, amount of fucosylation, amount of sialylation, glycan site occupancy, or level of high mannose glycans, wherein the value can be qualitative (present or absent) or quantitative. A target glycan structure can refer to the glycan component at a single N-linked or O-linked glycosylation site on a molecule or to the glycan components at that site in a plurality, e.g., a purified preparation, of molecules (the target glycan structure of a glycan component). Thus, an example of a selected or specific glycan structure can be having monogalactosylation at a site, or a selected or specific level or percentage of monogalactosylation at that site in a plurality of molecules. (This is a target glycan structure for a glycan component.) A target glycan structure can refer collectively to the glycan components at two or more selected sites (N-linked or O-linked or a combination thereof) on a molecule or, at those sites in a plurality of molecules. Thus, an example of a selected or specific glycan structure can be monogalactosylation at a first and second site or a selected or specific level or percentage of monogalactosylation at those sites in a plurality, e.g., a purified preparation, of molecules. (This is a target glycan structure for a component of the glycan complement.) A target glycan structure can refer to the glycan components at all of the N-linked or O-linked glycosylation sites, or all sites, on a molecule or in a plurality of molecules. Thus, an example of a selected or specific glycan structure can be monogalactosylation or a selected or specific level or percentage of monogalactosylation in a plurality, e.g., a purified preparation, of molecules. (This is a target glycan structure for the glycan complement.)

Exemplary target glycan structures are described below.

1. A target glycan structure characterized by having a preselected level or amount one or more monosaccharides or glycans. The level can be, e.g., increased or decreased in comparison to a standard, e.g., what would be seen in the absence of the relevant manipulation. For example, a target glycan structure may be characterized by a specific molar ratio of a monosaccharide (e.g., fucose) to protein, e.g., a ratio of 1.9 mol fucose/mol protein, a ratio of 1.0 mol fucose/mol protein, or a ratio of 0.5 mol fucose/mol protein.

2. A target glycan structure having a preselected proportion or ratio of a first monosaccharide or glycan to a second monosaccharide or glycan. The proportion or ratio can be, e.g., increased or decreased in comparison to a standard, e.g., what would be seen in the absence of the relevant manipulation. Examples include:

a) a preselected proportion or ratio of monogalactosylated to digalactosylated glycans:

GalGlcNAc₂Man₃GlcNAc₂Fuc-Asn vs. Gal₂GlcNAc₂Man₃GlcNAc₂Fuc-Asn,

represented pictorially as:

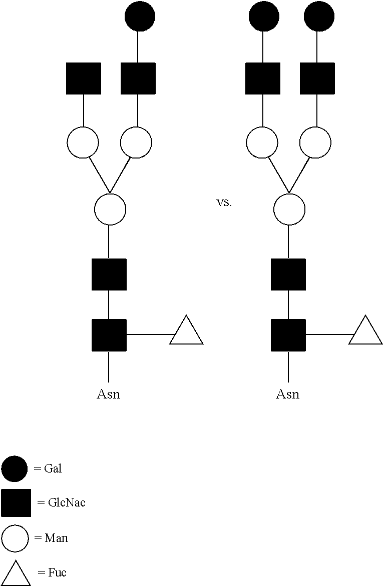

b) a preselected proportion or ratio of unfucosylated to fucosylated glycans:

Man₃GlcNAc₂-Asn vs. Man₃GlcNAc₂Fuc-Asn,

represented pictorially as:

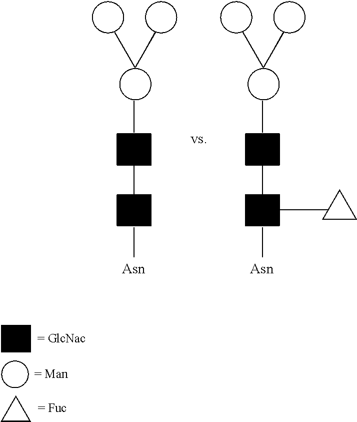

Fucose can be added at various points in the diversification process. This is just one possibility for the glycan structure with and without fucose.

3. A target glycan structure having: preselected heterogeneity or microheterogenity at a potential glycosylation site or across the entire protein, e.g., the degree of occupancy of potential glycosylation sites of a protein; structure of a branched (e.g., the presence, absence or amount of bisecting GlcNAc or mannose structures) or unbranched glycan; the presence, absence or amount of a glycan moiety (e.g., a complex (e.g., biantennary, triantennary, tetraantennary, etc.), a high mannose or a hybrid glycan moiety); relative position of a monosaccharide within a glycan chain (e.g., the presence, absence or amount of a terminal or penultimate chemical unit); chemical makeup of the target glycan structure (e.g. amounts and ratios or proportions of the monosaccharides in a particular target glycan structure). A parameter can be, e.g., increased or decreased in comparison to a standard, e.g., what would be seen in the absence of the relevant manipulation. In embodiments a target glycan structure can, by way of example, be released as a peak or other fraction (representing one or more species), e.g., from an enzymatic digest. A target glycan structure can be described, e.g., in defined structural terms, e.g., by chemical name, or by a functional or physical property, e.g., by molecular weight or by a parameter related to purification or separation, e.g., retention time of a peak in a column or other separation device.

In embodiments the target glycan structure is present with glycan components of other structures in a preparation of glycoprotein molecules. E.g., a selected position may be occupied by glycan components having different glycan structures in a preparation of glycoproteins. The abundance of a target glycan structure can be represented by the amount (by number or molecular weight) of that target glycan structure relative to other glycan components at that position(s) in a plurality of molecules. Methods disclosed herein can be used to alter the amount of a glycan component having a specific or selected glycan structure relative to other glycan components present in the preparation at the selected position. The amount of a target glycan structure in a preparation of glycoproteins can be expressed as increased or decreased. In an embodiment this means increased or decreased in comparison with a preselected standard, e.g., increased as compared to a standard wherein the target glycan structure is present at a preselected ratio or proportion, e.g., 1:1 with respect other glycan structures at the selected position. In embodiments increased or decreased means that the amount (or ratio or proportion) of a target glycan structure in a preparation of glycoproteins subjected to a manipulation is increased or decreased relative to the amount (or ratio or proportion) of a target glycan structure in a preparation of glycoproteins not subjected to the manipulation (in other words, a ratio of proportions).

### Regulation of Glycosylation

Glycosylation is a nonlinear non-template driven process. To this end, regulation of a particular glycan structure may be due to a number of orthogonal inputs such as precursor levels, donor levels, transferase levels to name a few. Glycosylation of proteins can have dramatic effect on their activities, such as regulating receptor affinity, regulating bioavailability, or altering immunogenicity.

Eukaryotic glycosylation occurs in the endoplasmic reticulum (ER) and Golgi through a stepwise process in which one monosaccharide is added through the activity of a glycosyltransferase, utilizing an activated sugar nucleotide as the donor molecule. The byproduct of the reaction, the nucleoside diphosphate, is then metabolized by a specific nucleoside diphosphatase, and the product nucleoside monophosphate is pumped out of the Golgi into the cytoplasmic milieu. The activity of the glycosyltransferase can be regulated by ionic strength, divalent cations, as well as the presence of nucleoside monophosphates and diphosphates. The nucleoside phosphates act as competitive inhibitors against the normal sugar-nucleotide substrates. The pump, however, is thought to be bidirectional; it pumps nucleoside monophosphates out of the Golgi and into the cytoplasm and pumps nucleoside diphosphates from the cytoplasm into the Golgi.

The graphic below illustrates this with a UDP sugar.

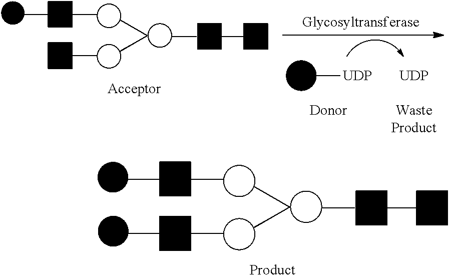

Methods of regulating cellular glycosylation by controlling the levels of the nucleoside diphosphate available are disclosed herein. It is surprising that this regulation would be effective in light of the bidirectional nature of the pump results in a background of influx of nucleoside diphosphates. Methods of regulating the Golgi guanosine diphosphatase or the Golgi uridine diphosphatase to regulate the levels of the diphosphate inhibitors and thus the activity of glycosyltransferases are disclosed herein. In some embodiments this may involve the use of phosphatase inhibitors or by way of genetic manipulation to alter the activity or specificity of the nucleoside diphosphatase. In other embodiments this may involve supplementation of the growth medium with a nucleoside diphosphate or similar derivative.

### Glycosyltransferases

Glycosyltransferases are enzymes that catalyze the transfer of a monosaccharide from an activated sugar donor (known as the “glycosyl donor”) to an acceptor molecule. Glycosyl transfer can occur directly to a protein side chain, or to a preexisting glycan on a glycoprotein. The result of glycosyl transfer can be a monosaccharide glycoside, an oligosaccharide, or a polysaccharide.

Most commonly, sugar-nucleotide derivatives are used as glycosyl donors. Examples of common sugar nucleotide donors include those attached to uridine diphosphate (UDP), such as UDP-glucose, UDP-galactose, UDP-N-acetylglucosamine, and UDP-N-acetylgalactosamine; and those attached to guanosine diphosphate (GDP), such as GDP-mannose and GDP-fucose. Following glycosyltransferase-catalyzed sugar transfer from the activated sugar donor to the acceptor, the byproduct of the reaction is the corresponding nucleoside diphosphate (e.g., UDP or GDP). This byproduct may inhibit the glycosyltransferase, as described above.

### Nucleoside Diphosphatases

Nucleoside diphosphatases are enzymes that catalyze the hydrolysis of nucleoside diphosphates to the corresponding nucleoside monophosphates and inorganic phosphate. These enzymes are found in the Golgi and serve to remove the nucleoside diphosphates that are inhibitory byproducts of the reactions catalyzed by glycosyltransferases (see above). For example, uridine diphosphatase hydrolyzes UDP to UMP according to Scheme 1. Once the nucleoside diphosphate is hydrolyzed, the resulting nucleoside monophosphate is exported out of the Golgi. These enzymes play an important role in removing the inhibitory NDP byproduct of the glycosyltransferase reaction.

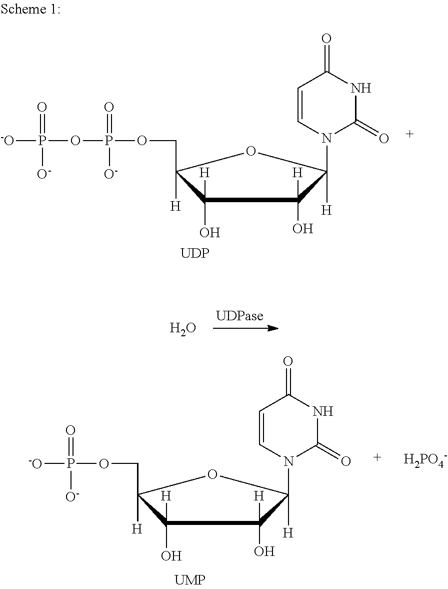

Nucleoside diphosphatases used in the methods and cells described herein include mammalian, e.g., human, mouse, rat or hamster, nucleoside diphosphatases. The nucleoside diphosphatase can be a primate, e.g., a human, nucleoside diphosphatase. In other embodiments the nucleoside diphosphatase is a rodent nucleoside diphosphatase, e.g., a mouse, rat or hamster, nucleoside diphosphatase.

A nucleoside diphosphatase sequence, e.g., a nucleoside diphosphatase encoding sequence, can be used to increase or decrease the nucleoside diphosphatase expression in a cell. For example, expression can be increased by adding an additional copy of a nucleoside diphosphatase to the cell, or by use of a nucleoside diphosphatase sequence to place the endogenous nucleoside diphosphatase under the control of a regulatory element that increases expression (e.g., use of a nucleoside diphosphatase homologous sequence to insert an up-regulating regulatory element, e.g., an enhancer or promoter, into the genome and thereby up-regulate the endogenous nucleoside diphosphatase). A decrease in nucleoside diphosphatase expression can be achieved by inactivating the endogenous nucleoside diphosphatase gene, e.g., in the control or structural regions. A cloned nucleoside diphosphatase sequence can be used to make a construct that will insert a deletion or other event into an endogenous gene to decrease levels of the protein it expresses. The decrease can be partial, in other words, activity is not wholly eliminated, e.g., in comparison with a gene or strain that does not have the manipulation, or complete. In an embodiment manipulation is a genetic manipulation, e.g., a mutation, which decreases, e.g., partially or completely, the level of activity of uridine diphosphatase in the product of the manipulated uridine diphosphatase gene.

The expression of endogenous nucleoside diphosphatase can be increased or decreased by the use of a genetic construct from the same species as the endogenous nucleoside diphosphatase, or from a different species. For example, the expression of an endogenous nucleoside diphosphatase in a mouse cell can be modulated with a construct made from mouse nucleoside diphosphatase or with one made from a nucleoside diphosphatase sequence from another species, e.g., a different rodent species. The nucleoside diphosphatase of a rodent, e.g., a hamster, such as a Chinese hamster, can be manipulated with an allogeneic sequence (from the same species) or a xenogeneic sequence (from a different species). For example, a CHO cell can be manipulated with a Chinese hamster, mouse or rat sequence.

A nucleic acid sequence from one of the nucleoside diphosphatases disclosed herein can be used to isolate a nucleoside diphosphatase gene from a different species. For example, a mouse or rat sequence described herein can be used to make primers to isolate a nucleoside diphosphatase sequence from another rodent, e.g., a hamster, e.g., a Chinese hamster. That sequence can them be used to modify nucleoside diphosphatase expression in a cell, e.g., in a Chinese hamster cell, such as a CHO cell.

Exemplary nucleoside diphosphatases include the following:

### Protein Sequence of Human Uridine Diphosphatase (Ectonucleoside Triphosphate Diphosphohydrolase 4):

(SEQ ID NO: 1)
MGRIGISCLFPASWHFSISPVGCPRILNTNLRQIMVISVLAAAVSLLYFS
VVIIRNKYGRLTRDKKFQRYLARVTDIEATDTNNPNVNYGIVVDCGSSGS
RVFVYCWPRHNGNPHDLLDIRQMRDKNRKPVVMKIKPGISEFATSPEKVS
DYISPLLNFAAEHVPRAKHKETPLYILCTAGMRILPESQQKAILEDLLTD
IPVHFDFLFSDSHAEVISGKQEGVYAWIGINFVLGRFEHIEDDDEAVVEV
NIPGSESSEAIVRKRTAGILDMGGVSTQIAYEVPKTVSFASSQQEEVAKN
LLAEFNLGCDVHQTEHVYRVYVATFLGFGGNAARQRYEDRIFANTIQKNR
LLGKQTGLTPDMPYLDPCLPLDIKDEIQQNGQTIYLRGTGDFDLCRETIQ
PFMNKTNETQTSLNGVYQPPIHFQNSEFYGESEFYYCTEDVLRMGGDYNA
AKFTKAAKDYCATKWSILRERFDRGLYASHADLHRLKYQCFKSAWMFEVF
HRGFSFPVNYKSLKTALQVYDKEVQWTLGAILYRTRFLPLRDIQQEAFRA
SHTHWRGVSFVYNHYLFSGCFLVVLLAILLYLLRLRRIHRRTPRSSSAAA
LWMEEGLPAQNAPGTL
GenBank Accession No. NP_004892
(GenBank version dated 24-OCT-2008)

cDNA Sequence of Human Uridine Diphosphatase

(SEQ ID NO: 2)
ATGGGGAGGATTGGCATCTCCTGTCTTTTTCCTGCTTCTTGGCATTTTAG
CATATCTCCAGTAGGGTGTCCTCGAATTCTGAATACCAATTTACGCCAAA
TTATGGTCATTAGTGTCCTGGCTGCTGCTGTTTCACTTTTATATTTTTCT
GTTGTCATAATCCGAAATAAGTATGGGCGACTAACCAGAGACAAGAAATT
TCAAAGGTACCTGGCACGAGTTACCGACATTGAAGCTACAGACACCAATA
ACCCCAATGTGAACTATGGGATCGTGGTGGACTGTGGTAGCAGTGGGTCT
CGAGTATTTGTTTACTGCTGGCCAAGGCATAATGGCAATCCACATGATCT
GTTGGATATCAGGCAAATGAGGGATAAAAACCGAAAGCCAGTGGTCATGA
AGATAAAACCGGGCATTTCAGAATTTGCTACCTCTCCAGAGAAAGTCAGT
GATTACATTTCTCCACTTTTGAACTTTGCTGCAGAGCATGTGCCACGGGC
AAAACACAAAGAGACACCTCTCTACATTCTCTGCACGGCTGGAATGAGAA
TCCTCCCCGAAAGCCAGCAGAAAGCTATTCTGGAAGACCTTCTGACCGAT
ATCCCCGTGCACTTTGACTTTCTGTTTTCTGACTCTCATGCAGAAGTAAT
TTCTGGGAAACAAGAAGGTGTGTATGCTTGGATTGGCATTAATTTTGTCC
TTGGACGATTTGAGCATATTGAAGATGATGATGAGGCCGTTGTGGAAGTT
AACATTCCTGGAAGTGAAAGCAGCGAAGCCATTGTCCGTAAAAGGACAGC
GGGCATTCTCGACATGGGCGGCGTGTCGACTCAGATAGCGTACGAAGTCC
CCAAAACTGTAAGCTTTGCGTCCTCACAGCAGGAAGAAGTAGCTAAAAAC
TTGTTAGCTGAATTTAACTTGGGATGTGATGTTCACCAAACTGAGCATGT
GTATCGAGTCTATGTGGCCACGTTTCTTGGGTTTGGTGGCAATGCTGCTC
GACAGAGATACGAAGACAGAATATTTGCCAACACCATTCAAAAGAACAGG
CTCCTGGGTAAACAGACTGGTCTGACTCCTGATATGCCGTACTTGGACCC
CTGCCTACCCCTAGACATTAAAGATGAAATCCAGCAAAATGGACAAACCA
TATACCTACGAGGGACTGGAGACTTTGACCTGTGTCGAGAGACTATCCAG
CCTTTCATGAATAAAACAAACGAGACCCAGACTTCCCTCAATGGGGTCTA
CCAGCCCCCAATTCACTTCCAGAACAGTGAATTCTATGGCTTCTCCGAAT
TCTACTACTGCACCGAGGATGTGTTACGAATGGGGGGAGACTACAATGCT
GCTAAATTTACTAAAGCTGCAAAGGATTATTGTGCAACAAAGTGGTCCAT
TTTGCGGGAACGCTTTGACCGAGGACTGTACGCCTCTCATGCTGACCTCC
ACAGGCTTAAGTATCAGTGCTTCAAATCGGCCTGGATGTTTGAGGTGTTT
CATAGGGGCTTTTCGTTTCCTGTCAACTATAAAAGCTTAAAGACTGCCTT
GCAAGTTTACGACAAGGAGGTTCAGTGGACCCTTGGAGCCATCCTCTACA
GGACCCGCTTTCTACCATTAAGAGACATCCAGCAGGAGGCCTTCCGAGCC
AGTCACACCCACTGGCGGGGCGTTTCCTTTGTCTACAACCACTACCTGTT
CTCTGGCTGCTTCCTGGTGGTGCTGCTGGCCATCCTGCTGTACCTGCTGC
GGCTGCGGCGCATCCACAGGCGCACTCCCCGGAGCAGCTCGGCCGCCGCC
CTCTGGATGGAGGAGGGCCTTCCCGCCCAGAATGCCCCGGGGACCTTGTG
A
GenBank Accession No. NM_004901
(GenBank version dated 24-OCT-2008)

### Protein Sequence of Murine Uridine Diphosphatase

(SEQ ID NO: 3)
MGRIGISCLFPASWHFSISPVGCPRILNTNLRQIVVISILAAAVSLLYFS
VVIIRSKYGWLSKDKKFQRYLARVTDVEATDTNNPSVNYGIVVDCGSSGS
RIFVYCWPRHNGNPHDLLDIRQMRDKNRKPVVMKIKPGISEFATSPEKVS
DYISPLLSFAAEHVPRAKHKETPLYILCTAGMRVLPESQQKAILEDLLTD
IPVHYDFLFSDSHAEVISGKQEGVYAWIGINFVLGRFEHIEEDDEAVVEV
NIPGSESSEAIVRKRTAGVLDMGGVSTQIAYEVPQTVSFASSQQEEVAKN
LLAEFNLGCDVHQTEHVYRVYVATFLGFGGNAARQRYEDRLFASTVQKNR
LLGKQTGLTPDAPLLDPCLPLDIKDEIQQNGQTLYLQGTGDFDLCRETLQ
PFMNKTNETQTSLNGVYQPPIHFQNSEFYGFSEFYYCTEDVLRMGGDYNA
ARFTQAAKDYCATKWSILRERFDRGLYASHADLHRLKYQCFKSAWMFEVF
HKGFSFPVTYKNLKTALQVYDKEVQWTLGAILYRTRFLPLRDIRQEVFRA
GHAHWRGVSFVYNHYLFSGCFLVVLLSILLYLLRLRRIHRRAPRTGSLWM
EEGLPSQKGPGPL
GenBank Accession No. NP_0804500
(GenBank version dated 24-OCT-2008)

cDNA Sequence Murine Uridine Diphosphatase

(SEQ ID NO: 4)
ATGGGGAGGATTGGCATTTCCTGTCTCTTTCCTGCCTCTTGGCATTTTAG
CATCTCTCCAGTGGGCTGTCCTCGAATTCTGAACACCAATTTACGACAAA
TCGTTGTCATTAGCATCCTGGCTGCAGCTGTCTCCCTTTTATACTTCTCT
GTTGTCATAATCCGCAGCAAGTATGGGTGGCTGTCAAAGGACAAGAAATT
TCAAAGGTACTTGGCCCGAGTCACAGACGTTGAGGCCACAGACACCAACA
ACCCCAGCGTGAACTATGGCATCGTGGTGGACTGCGGCAGCAGTGGGTCT
CGGATATTTGTCTACTGCTGGCCTCGGCACAATGGCAACCCTCACGATCT
GCTGGACATCAGACAGATGAGGGACAAAAACCGGAAGCCAGTGGTGATGA
AGATTAAGCCCGGCATCTCAGAGTTTGCTACCTCTCCAGAAAAAGTCAGC
GACTACATTTCTCCGCTTCTGAGCTTTGCTGCAGAACATGTGCCTCGGGC
AAAACACAAAGAGACACCTCTCTACATTCTCTGCACAGCTGGAATGAGAG
TCCTTCCTGAAAGCCAGCAGAAAGCCATCCTAGAGGACCTCCTGACCGAC
ATCCCTGTGCACTATGATTTCCTGTTTTCTGACTCCCATGCCGAAGTCAT
CTCAGGAAAACAAGAAGGTGTGTATGCTTGGATCGGCATTAATTTTGTCC
TCGGACGGTTTGAGCATATTGAGGAGGATGACGAGGCGGTTGTGGAAGTC
AACATTCCGGGCAGCGAGAGCAGCGAGGCCATCGTGCGGAAAAGGACAGC
TGGTGTCCTCGACATGGGAGGCGTGTCTACCCAGATAGCGTACGAAGTCC
CCCAAACTGTAAGCTTTGCCTCCTCGCAGCAGGAAGAAGTAGCTAAAAAC
CTGTTAGCTGAATTCAACCTGGGGTGCGATGTCCACCAGACTGAGCATGT
GTACCGCGTCTACGTGGCCACGTTTCTTGGGTTTGGTGGTAATGCTGCCC
GGCAGAGATATGAAGACCGACTATTTGCCAGCACAGTTCAGAAAAACAGG
CTCCTGGGTAAACAGACTGGTCTGACTCCTGATGCTCCACTACTGGATCC
CTGCTTGCCTCTGGACATTAAAGATGAGATCCAGCAAAACGGGCAGACCC
TGTACCTTCAGGGGACAGGAGACTTTGACCTGTGTCGAGAGACCCTGCAG
CCTTTCATGAACAAAACCAATGAGACCCAGACTTCCCTCAATGGCGTCTA
CCAGCCTCCAATCCACTTCCAGAACAGTGAATTCTACGGCTTCTCTGAGT
TCTACTACTGCACCGAGGATGTCTTGCGAATGGGGGGAGACTACAATGCT
GCTAGATTCACTCAAGCTGCCAAGGATTACTGTGCAACAAAGTGGTCGAT
CCTGCGGGAACGCTTTGACCGAGGACTCTATGCCTCTCATGCCGACCTCC
ATCGACTGAAGTATCAGTGTTTCAAATCAGCCTGGATGTTCGAGGTGTTC
CACAAAGGCTTCTCCTTTCCTGTCACCTACAAAAACCTGAAGACGGCCTT
GCAGGTGTATGACAAGGAAGTACAGTGGACCCTGGGGGCCATCCTTTACC
GGACCCGCTTCCTGCCCTTGAGAGACATCCGGCAGGAGGTGTTCCGAGCT
GGCCACGCGCACTGGCGGGGCGTGTCCTTCGTCTACAACCACTATCTGTT
CTCCGGTTGCTTCCTGGTCGTCCTTCTGTCCATCCTTCTCTACCTGCTGC
GGCTGCGGCGCATCCACCGCAGGGCGCCCCGCACTGGCTCTCTGTGGATG
GAGGAAGGCCTGCCCTCCCAGAAGGGCCCTGGGCCCTTGTGA
GenBank Accession No. NM_026174
(GenBank version dated 24-OCT-2008)

### Protein Sequence of Rat Uridine Diphosphatase

(SEQ ID NO: 5)
MGSISPVGCPRILNTNLRQIVVISILAAAVSLLYFSVVIIRSKYGWLSKD
KKFQRYLARVTDVEATDTNNPNVNYGIVVDCGSSGSRIFVYCWPQHNGNP
HDLLDIRQMRDKNRKPVVMKIKPDEIQQNGQTLYLRGTGDFDLCRETLQP
FMNKTNETQTSLNGVYQPPIHFQNSEFYGFSEFYYCTEDVLRMGGDYNAA
KFTKAAKDYCATKWSILRERFDRGLYASHADLHRLKYQCFKSAWMFEVFH
RGFSFPVTYKSLKTALQVYDKEVQWTLGAILYRTRFLPLRDIRQEVFRAG
HAHWQGVSFVYNHYLFSGCFLVVLLSILLYLLRLRRIHRRAPRTGSLWME
EGLPSQKGPGPL
GenBank Accession No. NP_001101854
(GenBank version dated 24-OCT-2008)

mRNA Sequence of Rat Uridine Diphosphatase

(SEQ ID NO: 6)
ccctacgtgcgcgcgccggcgcgagttgtgacgtgacgttggcgggcgcgcgcagcgtgactcccgaaggagccgaacctc
cgcaaagctggtggccgggatgcggtgcgctattggccgcccgctcccccggagccgcggcccgcccagcagggtagctct
gactccatgaagaccccagctccgattctgtcattgtagatgacgagaactgaatcccacaacattgcctggaccttgcttggcctt
tcagtATGGGGAGCATCTCGCCAGTGGGCTGTCCTCGAATTCTGAACACCAATTT
ACGACAAATCGTTGTCATTAGCATCCTGGCTGCAGCTGTCTCCCTTTTATACT
TTTCTGTTGTCATAATCCGCAGCAAGTATGGGTGGCTGTCAAAGGACAAGAA
ATTTCAAAGGTACTTGGCCCGAGTCACAGACGTTGAGGCTACAGACACCAAC
AACCCCAACGTGAACTATGGCATTGTGGTGGACTGCGGCAGTAGTGGGTCTC
GGATATTTGTCTATTGCTGGCCTCAGCACAACGGCAATCCTCATGACCTGCTG
GACATCAGACAGATGAGGGACAAAAACCGGAAGCCAGTGGTGATGAAAATT
AAGCCCGATGAGATCCAGCAAAATGGGCAAACCCTGTACCTTCGGGGGACA
GGAGACTTCGACCTGTGTCGAGAGACCCTCCAGCCTTTCATGAACAAAACCA
ATGAGACACAGACTTCTCTCAATGGAGTCTACCAGCCTCCAATCCACTTCCAG
AACAGTGAATTCTATGGCTTCTCTGAGTTCTACTATTGCACCGAAGATGTCTT
ACGAATGGGGGGAGACTACAATGCTGCTAAATTTACTAAAGCTGCCAAGGAT
TACTGTGCAACAAAGTGGTCGATCTTGCGGGAACGCTTTGACCGAGGACTGT
ACGCCTCTCATGCCGACCTCCATCGACTTAAGTATCAGTGTTTCAAATCAGCC
TGGATGTTTGAGGTGTTCCACAGGGGCTTCTCCTTCCCTGTCACATACAAAAG
TCTGAAGACAGCCTTGCAGGTGTATGACAAGGAAGTGCAGTGGACCCTGGGG
GCAATCCTTTACAGGACCCGCTTTCTGCCCTTGAGAGACATCCGACAGGAGG
TGTTCCGGGCTGGCCATGCACACTGGCAGGGCGTGTCCTTTGTCTACAACCAC
TATCTGTTCTCTGGTTGCTTCCTGGTCGTCCTTCTATCCATCCTTCTCTACCTGC
TGCGGCTTCGGCGCATCCACCGCAGGGCACCCCGCACTGGCTCTCTGTGGAT
GGAGGAAGGCCTGCCCTCCCAGAAGGGCCCTGGGCCCTTGTGAcagacactgtgtcag
cttgaagaagactctacaggaaaagccatttttgcctcagggtttctcatatgctccaattgttttgtttgtccctttcctttctgttacaa
aaccccactgatttgtaaaccctgctgtctagaggtactaccattttgaacgcagcttaaaatggaggagtggaaaagagacttca
ctcagcttgtgctgcacagcatctgccacacatcagtaaggtttgtaggaagtgctgcatttttagcacatgcacttgggtacatgc
acaaggggacagtgggcaagttcccatcagcgtagatggaacttcagagtggtcctgggacagaaccaagctgtgagttttacc
ctctttcctctccaaacacctcaagctagaaggggggcgtttgattttatttgctgctcaggtctgtcaccatctgtgttttcttggcag
atttaagactttagtcatttatagcaaaaatcgacaagatggtgcacagggaggtgatacgaaagaggggtcagtgatgagaact
actgaggagaacattgccctgctgcaggcgatcgcatgcctgtaaactagccgcacttgcccctgtgctggggagctctgtcgc
cctcttagagcagcagtgagtttgtttgagtgctcatttgttttatttgtttgtttgtttttaaaccagaaagtctataaagttcccaggttta
gtggtctgagagcgtggaccaggagtatgccctgcaggcacccagtacctctgagaggcaggtctgtgctgtcgagctgcccc
agcctcttccacttcctgtgtcacccccatggttcagatctcttcactgtctttcttcaggacaccccacacattgctcgacagtcctt
gttgtcacactgtggctgcagctgtcgctggcagtggcactgtaagcccacaccgtggaagagcctgaatttaaaataagaaata
aatgcacacgttgaaaacaaatttgacattttaagtggaaacctgaaaaggacaaccggggatatgcggggctg
GenBank Accession No. NM_001108384 (GenBank version dated 24-OCT-2008)

### Manipulations

As described above, a manipulation, as used herein, refers to a property of a cell.

One example of a manipulation is a nucleoside diphosphatase activity (NDPA manipulations). Such manpulations include the presence in or on the cell of an exogenous inhibitor of a nucleoside diphosphatase such a nucleic acid antagonist (e.g., an siRNA), or a chemical agent (e.g., a phosphatase inhibitor). Examples of NDPA manipulations also include a mutation or other genetic event, e.g., a genetically engineered knock out, that inhibits or enhances the level of nucleoside diphosphatase activity.

A manipulated cell can be, e.g., a vertebrate, mammalian or rodent cell. Primers or other nucleic acids used, e.g., to form or make manipulations, can be, e.g., vertebrate, mammalian or rodent sequences. For example, a rodent primer or other nucleic acid, e.g., a nucleic acid encoding an active or inactivate rodent nucleoside diphosphatase, can be used to manipulate a rodent cell. Similarly, a mammalian cell having a manipulation can be made with mammalian nucleic acids, e.g., mammalian primers or a nucleic acid encoding a mammalian nucleoside diphosphatase. A sequence from a first species can be used to manipulate a cell of a second species. E.g., a primer or nucleic acid from a first species, e.g., a first rodent species, e.g., a mouse or rat, can be used to manipulate a cell from a second species, e.g., a second rodent species, e.g., a hamster cell, e.g., a CHO cell.

Nucleic Acid Antagonists

In some embodiments, nucleic acid antagonists are used to decrease expression of a target protein, e.g., a protein involved in regulating nucleoside diphosphate levels, e.g., a nucleoside diphosphatase. In one embodiment, the nucleic acid antagonist is an siRNA that targets mRNA encoding the target protein. Other types of antagonistic nucleic acids can also be used, e.g., a nucleic acid aptamer, a dsRNA, a ribozyme, a triple-helix former, or an antisense nucleic acid.

siRNAs can be used to inhibit expression of nucleoside diphosphatases. siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically the siRNA sequences are exactly complementary to the target mRNA. dsRNAs and siRNAs in particular can be used to silence gene expression in mammalian cells (e.g., human cells). See, e.g., Clemens, J. C. et al. (2000) Proc. Natl. Sci. USA 97, 6499-6503; Billy, E. et al. (2001) Proc. Natl. Sci. USA 98, 14428-14433; Elbashir et al. (2001) Nature 411(6836):494-8; Yang, D. et al. (2002) Proc. Natl. Acad. Sci. USA 99, 9942-9947, US 2003-0166282, 2003-0143204, 2004-0038278, and 2003-0224432.

Anti-sense agents can also be used to inhibit expression of nucleoside diphosphatases and include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides that hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired.

Hybridization of antisense oligonucleotides with mRNA can interfere with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity that may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N4-(C1-C12)alkylaminocytosines and N4,N4-(C1-C12)dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N6-(C1-C12)alkylaminopurines and N6,N6-(C1-C12)dialkylaminopurines, including N6-methylaminoadenine and N6,N6-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. No. 4,987,071; U.S. Pat. No. 5,116,742; U.S. Pat. No. 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher, L. J. (1992) Bioassays 14:807-15.

Phosphatase Inhibitors

Phosphatase inhibitors are molecules that bind to phosphatases and decrease their activities. The binding of an inhibitor may stop a substrate from entering the phosphatase active site and/or hinder the phosphatase from catalyzing its reaction Inhibitor binding may be either reversible or irreversible. Irreversible inhibitors usually react with the enzyme and change it chemically. These inhibitors modify key amino acid residues needed for phosphatase activity. In contrast, reversible inhibitors bind non-covalently and different types of inhibition are produced depending on whether these inhibitors bind the phosphatase, the phosphatase-substrate complex, or both.

A phosphatase inhibitor may be a broad-spectrum phosphatase inhibitor. Many such inhibitors are commercially available and include β-glycerophosphate, (−)-p-bromotetramisole oxalate, EDTA, sodium fluoride, sodium molybdate, sodium orthovanadate, sodium pyrophosphate and sodium tartrate.

A phosphatase inhibitor may also be a specific substrate or product analog that can be used to inhibit a nucleoside diphosphatase. Examples of such substrate or product analogs include uridine-5′-O-thiophosphate, uridine-5′-O-(2-thiodiphosphate), uridine-5′-O-(3′-thiotriphosphate), guanosine-5′-O-thiophosphate, guanosine-5′-O-(2-thiodiphosphate), guanosine-5′-O-(3′-thiotriphosphate), cytosine-5′-O-thiophosphate, cytosine-5′-O-(2-thiodiphosphate), and cytosine-5′-O-(3′-thiotriphosphate).

Phosphatase inhibitors may be evaluated to determine whether they inhibit a nucleoside diphosphatase. The inhibitors may be evaluated in vitro or in vivo. For example, a cell or population of cells may be treated with the inhibitor of interest. A preparation of a glycoprotein or a group of glycoproteins may then be analyzed using a method described herein, to determine if the glycan has been altered.

The evaluation of phosphatase inhibitors may also include an analysis of potential toxicity to the cell or population of cells.

Genetically Engineered Knock Out Cells

In some embodiments, a cell can be selected that has been genetically engineered for permanent or regulated inactivation of a gene encoding a nucleoside diphosphatase, or a protein involved in regulating nucleoside diphosphate levels. For example, genes encoding a nucleoside diphosphatase described herein can be inactivated. Permanent or regulated inactivation of gene expression can be achieved by targeting to a gene locus with a transfected plasmid DNA construct or a synthetic oligonucleotide. The plasmid construct or oligonucleotide can be designed to several forms. These include the following: 1) insertion of selectable marker genes or other sequences within an exon of the gene being inactivated; 2) insertion of exogenous sequences in regulatory regions of non-coding sequence; 3) deletion or replacement of regulatory and/or coding sequences; and, 4) alteration of a protein coding sequence by site specific mutagenesis.

In the case of insertion of a selectable marker gene into a coding sequence, it is possible to create an in-frame fusion of an endogenous exon of the gene with the exon engineered to contain, for example, a selectable marker gene. In this way following successful targeting, the endogenous gene expresses a fusion mRNA (nucleic acid sequence plus selectable marker sequence). Moreover, the fusion mRNA would be unable to produce a functional translation product.

In the case of insertion of DNA sequences into regulatory regions, the transcription of a gene can be silenced by disrupting the endogenous promoter region or any other regions in the 5′ untranslated region (5′ UTR) that is needed for transcription. Such regions include, for example, translational control regions and splice donors of introns. Secondly, a new regulatory sequence can be inserted upstream of the gene that would render the gene subject to the control of extracellular factors. It would thus be possible to down-regulate or extinguish gene expression as desired for glycoprotein production. Moreover, a sequence that includes a selectable marker and a promoter can be used to disrupt expression of the endogenous sequence. Finally, all or part of the endogenous gene could be deleted by appropriate design of targeting substrates.

Cells Genetically Engineered to Express a Component Involved in Nucleoside Diphosphate Regulation

Cells can be genetically engineered to express a component involved in nucleoside diphosphate regulation, e.g., a cell can be genetically engineered to overexpress a nucleoside diphosphatase. When cells are to be genetically modified for the purposes of expressing or overexpressing a component, the cells may be modified by conventional genetic engineering methods or by gene activation.

According to conventional methods, a DNA molecule that contains cDNA or genomic DNA sequence encoding desired protein may be contained within an expression construct and transfected into primary, secondary, or immortalized cells by standard methods including, but not limited to, liposome-, polybrene-, or DEAE dextran-mediated transfection, electroporation, calcium phosphate precipitation, microinjection, or velocity driven microprojectiles (see, e.g., U.S. Pat. No. 6,048,729).

Alternatively, one can use a system that delivers the genetic information by viral vector. Viruses known to be useful for gene transfer include adenoviruses, adeno associated virus, herpes virus, mumps virus, pollovirus, retroviruses, Sindbis virus, and vaccinia virus such as canary pox virus.

Alternatively, the cells may be modified using a gene activation approach, for example, as described in U.S. Pat. No. 5,641,670; U.S. Pat. No. 5,733,761; U.S. Pat. No. 5,968,502; U.S. Pat. No. 6,200,778; U.S. Pat. No. 6,214,622; U.S. Pat. No. 6,063,630; U.S. Pat. No. 6,187,305; U.S. Pat. No. 6,270,989; and U.S. Pat. No. 6,242,218.

Accordingly, the term “genetically engineered,” as used herein in reference to cells, is meant to encompass cells that express a particular gene product following introduction of a DNA molecule encoding the gene product and/or including regulatory elements that control expression of a coding sequence for the gene product. The DNA molecule may be introduced by gene targeting or homologous recombination, i.e., introduction of the DNA molecule at a particular genomic site.

A component involved in nucleoside diphosphatase regulation of glycosylation, e.g., a nucleotide diphosphatase, can be placed under manipulable, e.g., inducible control. For example, a nucleoside diphosphatase encoding sequence can be placed under the control of a promoter or other control element that is responsive to an inducer (or inhibitor) of expression. Such systems allow the cell to be maintained under a variety of conditions, e.g., a condition wherein the gene, e.g., a gene encoding a nucleoside diphosphatase, is expressed or not expressed. This allows culture of the cell under a first condition, which provides glycoproteins having a first glycosylation state (e.g., wild-type), or under a second condition, which provides glycoproteins having a second glycosylation state (e.g., a glycosylation state described herein).

Cells can also be engineered to express a hybrid nucleic acid; that is, a nucleic acid comprising at least two segments which have been isolated from at least two different sources. As one example of manipulation of a cell with a hybrid nucleic acid, a mammalian cell having a manipulation may express a hybrid nucleic acid comprising a regulatory sequence, such as a promoter and/or terminator sequence, of mammalian cell origin, which is functionally linked to a coding sequence, which may be of origin from a different species, e.g., from a different mammal or non-mammalian. In this manner, for example, a cell may be manipulated so that it can be induced to express the coding sequence in response to a stimulus that does not naturally induce expression of the linked coding sequence. An example of such a system is the TET On/Off regulatory system. In the Tet-Off system, gene expression is turned on when tetracycline (Tc) or doxycycline (Dox; a Tc derivative) is removed from the culture medium. In contrast, expression is turned on in the Tet-On system by the addition of Dox. The Tet-On system is responsive only to Dox, not to Tc. Both systems permit gene expression to be tightly regulated in response to varying concentrations of Tc or Dox

Methods of transfecting cells, and reagents such as promoters, markers, signal sequences that can be used for recombinant expression are known.

Selected Functional Manipulations

Another example of a manipulation is a selected functional manipulation, which is a physical characteristic or property characterized by the process that gave rise to it, e.g., cell that was placed under selective conditions that result in the cell being able to produce a glycoprotein having a target glycan structure, wherein the underlying basis for the ability to produce said glycoprotein having a target glycan structure may or may not be known or characterized.

The selection can be based on one or more of: selection for the ability to produce a product having a target glycan structure; selection for increased or decreased nucleoside diphosphatase activity; or increased or decreased level of nucleoside diphosphatase.

### Glycoproteins

Glycoproteins that can be made by methods described herein include those in Table 1 below.

TABLE 1

| Protein Product | Reference Listed Drug |
|---|---|
| interferon gamma-1b | Actimmune ® |
| alteplase; tissue plasminogen activator | Activase ®/Cathflo ® |
| Recombinant antithemophilic factor | Advate |
| human albumin | Albutein ® |
| Laronidase | Aldurazyme ® |
| interferon alfa-N3, human leukocyte derived | Alferon N ® |
| human antithemphilic factor | Alphanate ® |
| virus-filtered human coagulation factor IX | AlphaNine ® SD |
| Alefacept; recombinant, dimeric fusion protein LFA3-Ig | Amevive ® |
| Bivalirudin | Angiomax ® |
| darbepoetin alfa | Aranesp ™ |
| Bevacizumab | Avastin ™ |
| interferon bta-1a; recombinant | Avonex ® |
| coagulation factor IX | Beneflex ™ |
| Interferon beta-1b | Betaseron ® |
| Tositumomab | BEXXAR ® |
| antithemphilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta-glucocerebrosidase | Ceredase ® |
| imiglucerase; recombinant form of beta-glucocerebrosidase | Cerezyme ® |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Ferinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Hellxate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor complex-human | Humate-P ® |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Keplivance |
| Anakinra | Kineret ™ |
| antithemophilic factor | Kogenate ®FS |
| insulin glargine | Lantus ® |
| granulocyte macrophage colony-stimulating factor | Leukine ®/Leukine ® Liquid |
| lutropin alfa for injection | Luveris |
| OspA lipoprotein | LYMErix ™ |
| Ranibizumab | LUCENTIS ® |
| gemtuzumab azogamicin | Mylotarg ™ |
| Galsulfase | Naglazyme ™ |
| Nestritide | Natrecor ® |
| Pegfilgrastim | Neulasta ™ |
| Oprelvekin | Neumega ® |
| Filgrastim | Neupogen ® |
| Fanolesomab | NeutroSpec ™ (formely LeuTech ®) |
| somatropin [rDNA] | Norditropin ®/Norditropin Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| line attenuated Bacillus Calmette-Guerin | Pacis ® |
| peginterferon alfa-2a | Pegasys ® |
| pegylated version of interferon alfa-2b | PEG-Intron ™ |
| Abarelix (inhectable suspension); gonadotropin-releasing | Plenaxis ™ |
| hormone antagonist | |
| epoletin alfa | Procrit ® |
| Aldesleukine | Proleukin, IL-2 ® |
| Somatrem | Protropin ® |
| dornase alfa | Pulmozyme ® |
| Efalizumab; selective, reversible T-cell blocker | RAPTIVA ™ |
| combination of ribavirin and alpha interferon | Rebetron ™ |
| Interferon beta 1a | Rebif ® |
| antithemophilic factor | Recombinate ® rAHF/ |
| antithemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | Secreflo ™ |
| Basiliximab | Simulect ® |
| Eculizumab | SOLARIS (R) |
| Pegvisomant | SOMAVERT ® |
| Palivizumab; remobinantly produced, humanized mAb | Synagis ™ |
| thyrotropin alfa | Thyrogn ® |
| Tenecteplase | TNKase ™ |
| Natalizumab | TYSABRI ® |
| human immune globulin intravenous 5% and 10% solutions | Venoglobulin-S ® |
| interferon alfa-n1, lymphoblastoid | Wellferon ® |
| drotecogin alfa | Xigris ™ |
| Omalizumab; recombinant DNA-derived humanized monoclonal | Xolair ® |
| antibody targeting immunoglobulin-E | |
| Daclizumab | Zenapax ® |
| ibritumomab tiuxetan | Zevalin ™ |
| Somatotropin | Zorbitive ™ (Serostim ®) |

Table 2 below shows examples of target glycan structures and their biological effects.

TABLE 2

| | | | Example |
|---|---|---|---|
| Glycan | Biology area | Rationale | Reference |
| Sialic acid | Bioavailability | Due to “blocking” the penultimate | Wasley et al. Blood |
| terminal | | sugar galactose so it is not recognized | (1991) 77 (12): |
| | | by hepatic lectin and cleared from | 2624-32 |
| | | circulation | |
| | Targeting | Potential for targeting to any class of | Collins et al. J. |
| | | sialic acid binding lectis which may | Immunol (2006) |
| | | include but are not limited to the | 177: 2994-3003 |
| | | selectins (E, P, and L) and the siglecs | |
| | | (1-11). | |
| | Receptor affinity | Receptor affinity may be attenuated or | Darling et al. |
| | | affected by the presence of a charged | Biochemistry |
| | | sialic acid moiety | 41(49) 14524-31 |
| Galactose | Bioavailability | A terminal galactose is recognized by | Wasley et al. Blood |
| terminal | | the asiologlycoprotein receptor (or | (1991) 77 (12): |
| | | hepatic lectin) and cleared from | 2624-32 |
| | | circulation | |
| | Targeting | Potential for targeting to or complex | Seymour et al. J. |
| | | with galactose binding proteins which | Clin Onc. (2002) |
| | | may include but are not limited to the | 20 (6) 1668 |
| | | galectins | |
| | C1q | C1q and complement cytotoxicity | |
| | | increases with galactose | |
| Alpha linked | Immunogenecity | C1q and complement cytotoxicity | Lavecchio et al |
| Galactose | | increases with galactose | Transplantation |
| terminal | | | (1995) 60 (8) 841- |
| | | | 7 |
| Fucosylation | ADCC | The presence of a core fucose moiety | Satoh et al. Exp. |
| | | decreases ADCC activity | Opin. Biol. Ther. |
| | | | (2006) 6 (11): |
| | | | 1161-73 |
| | Targeting | The presence of a branched fucose | Thomas et al. J. |
| | | moiety may be used to target the | Biol. Chem. (1999) |
| | | protein to specific lecting receptors | 274 (27) 19035- |
| | | which may include but are not limited | 40 |
| | | to the selectins (E, P, and L) | |
| High Mannose | Targeting | High mannose type structures | Grabowski Exp. |
| | | (including but not limited to Man5, | Opin. Drug Deliv. |
| | | Man6, Man7, Man8 and Man9) can be | (2006) 3 (6): 771 |
| | | used to target the protein to mannose | Takamatsu, |
| | | specific receptors (which may include | Glycocon. J. (2004) |
| | | but are not limited to the Macrophage | 20(6): 385-97 |
| | | mannose receptor) | |
| | | High-mannose FGF has different tissue | |
| | | distribution, specific distribution to | |
| | | kidney | |
| | Receptor affinity | High-mannose TSH showed the highest | Schaaf et al., Mol. |
| | | biopotency for cAMP and IP stimulation | Cell Endocrinol. |
| | | in CHO and and Cos-7 cells | (1997) 132(1- |
| | | | 20:185-94 |
| N-glycolyl | Immunogenecity | High levels of N-glycolyl neurmainic | |
| neuraminic acid | | acid may be immunogenic | |
| GlcNAc terminal | Bioavailability | Mediated through GlcNAc binding to | Jones et al. |
| | | Mannose receptor | Glycobiol epub |
| | | | PMID 17331977 |
| GlcNAc bisecting | Receptor affinity | Increased ADCC activity through higher | Davies et al. |
| | | affinity for Fc receptor | Biotech.Bioeng. |
| | | | (2001) 74:288- |
| | | | 294 |
| Site Occupancy | Receptor affinity/ | Potential to control receptor afinity. | Lund et al. J. |
| | function | Control complement mediated Ab | Immunol. (1996) |
| | | cytotoxicity | 157 4963-4969 |

### Analytical Methods

In general, a glycan preparation can be subjected to analysis to determine whether the glycan includes a particular type of structure (e.g., a glycan structure described herein). In some embodiments, the analysis comprises comparing the structure and/or function of glycans in one glycoprotein preparation to structure and/or function of glycans in at least one other glycoprotein preparation. In some embodiments, the analysis comprises comparing the structure and/or function of glycans in one or more of the samples to structure and/or function of glycans in a reference sample.

Structure and composition of glycans can be analyzed by any available method. In some embodiments, glycan structure and composition are analyzed by chromatographic methods, mass spectrometry (MS) methods, chromatographic methods followed by MS, electrophoretic methods, electrophoretic methods followed by MS, nuclear magnetic resonance (NMR) methods, and combinations thereof.

In some embodiments, glycan structure and composition can be analyzed by chromatographic methods, including but not limited to, liquid chromatography (LC), high performance liquid chromatography (HPLC), ultra performance liquid chromatography (UPLC), thin layer chromatography (TLC), amide column chromatography, and combinations thereof.

In some embodiments, glycan structure and composition can be analyzed by mass spectrometry (MS) and related methods, including but not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof.

In some embodiments, glycan structure and composition can be analyzed by electrophoretic methods, including but not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof.

In some embodiments, glycan structure and composition can be analyzed by nuclear magnetic resonance (NMR) and related methods, including but not limited to, one-dimensional NMR (1D-NMR), two-dimensional NMR (2D-NMR), correlation spectroscopy magnetic-angle spinning NMR(COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR(ROESY-NMR), nuclear overhauser effect spectroscopy (NOESY-NMR), and combinations thereof.

In some embodiments, techniques described herein may be combined with one or more other technologies for the detection, analysis, and or isolation of glycans or glycoproteins. For example, in certain embodiments, glycans are analyzed in accordance with the present disclosure using one or more available methods (to give but a few examples, see Anumula, Anal. Biochem. 350(1):1, 2006; Klein et al., Anal. Biochem., 179:162, 1989; and/or Townsend, R. R. Carbohydrate Analysis” High Performance Liquid Chromatography and Capillary Electrophoresis, Ed. Z. El Rassi, pp 181-209, 1995, each of which is incorporated herein by reference in its entirety). For example, in some embodiments, glycans are characterized using one or more of chromatographic methods, electrophoretic methods, nuclear magnetic resonance methods, and combinations thereof. Exemplary such methods include, for example, NMR, mass spectrometry, liquid chromatography, 2-dimensional chromatography, SDS-PAGE, antibody staining, lectin staining, monosaccharide quantitation, capillary electrophoresis, fluorophore-assisted carbohydrate electrophoresis (FACE), micellar electrokinetic chromatography (MEKC), exoglycosidase or endoglycosidase treatments, and combinations thereof. Those of ordinary skill in the art will be aware of other techniques that can be used to characterize glycans together with the methods described herein.

In some embodiments, methods described herein allow for detection of glycan structure (such as glycan structure described herein) that are present at low levels within a population of glycans. For example, the present methods allow for detection of glycan species that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, methods described herein allow for detection of particular structures (e.g., αglycan structure described herein) that are present at low levels within a population of glycans. For example, the present methods allow for detection of particular structures that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, methods described herein allow for detection of relative levels of individual glycan species within a population of glycans. For example, the area under each peak of a liquid chromatograph can be measured and expressed as a percentage of the total. Such an analysis provides a relative percent amount of each glycan species within a population of glycans. In another example, relative levels of individual glycan species are determined from areas of peaks in a 1D-NMR experiment, or from volumes of cross peaks from a ¹H-¹⁵N HSQC spectrum (e.g., with correction based on responses from standards), or by relative quantitation by comparing the same peak across samples.

In some embodiments, a biological activity of a glycoprotein preparation (e.g., a glycoprotein preparation) is assessed. Biological activity of glycoprotein preparations can be analyzed by any available method. In some embodiments, a binding activity of a glycoprotein is assessed (e.g., binding to a receptor). In some embodiments, a therapeutic activity of a glycoprotein is assessed (e.g., an activity of a glycoprotein in decreasing severity or symptom of a disease or condition, or in delaying appearance of a symptom of a disease or condition). In some embodiments, a pharmacologic activity of a glycoprotein is assessed (e.g., bioavailability, pharmacokinetics, pharmacodynamics). For methods of analyzing bioavailability, pharmacokinetics, and pharmacodynamics of glycoprotein therapeutics, see, e.g., Weiner et al., J. Pharm. Biomed. Anal. 15(5):571-9, 1997; Srinivas et al., J. Pharm. Sci. 85(1):1-4, 1996; and Srinivas et al., Pharm. Res. 14(7):911-6, 1997.

As would be understood to one of skill in the art, the particular biological activity or therapeutic activity that can be tested will vary depending on the particular glycoprotein or glycan structure.

The potential adverse activity or toxicity (e.g., propensity to cause hypertension, allergic reactions, thrombotic events, seizures, or other adverse events) of glycoprotein preparations can be analyzed by any available method. In some embodiments, immunogenicity of a glycoprotein preparation is assessed, e.g., by determining whether the preparation elicits an antibody response in a subject.

### Cells & Cell Lines

Methods described herein use cells to produce products having target glycan structures. Examples of cells useful in these and other methods described herein follow.

The cell useful in the methods described herein can be eukaryotic or prokaryotic, as long as the cell provides or has added to it the enzymes to activate and attach saccharides present in the cell or saccharides present in the cell culture medium or fed to the cells. Examples of eukaryotic cells include yeast, insect, fungi, plant and animal cells, especially mammalian cells. Suitable mammalian cells include any normal mortal or normal or abnormal immortal animal or human cell, including: monkey kidney CV1 line transformed by SV40 (COS-7, ATCC CRL 1651); human embryonic kidney line (293) (Graham et al., J. Gen. Virol. 36:59 (1977)); baby hamster kidney cells (BHK, ATCC CCL 10); Chinese Hamster Ovary (CHO), e.g., DG44, DUKX-V11, GS-CHO (ATCC CCL 61, CRL 9096, CRL 1793 and CRL 9618); mouse sertoli cells (TM4, Mather, Biol. Reprod. 23:243 251 (1980)); monkey kidney cells (CV1 ATCC CCL 70); African green monkey kidney cells (VERO-76, ATCC CRL 1587); human cervical carcinoma cells (HeLa, ATCC CCL 2); buffalo rat liver cells (BRL 3A, ATCC CRL 1442); human lung cells (W138, ATCC CCL 75); human liver cells (Hep G2, HB 8065); mouse melanoma cells (NSO); mouse mammary tumor (MMT 060562, ATCC CCL51), TRI cells (Mather, et al., Annals N.Y. Acad. Sci. 383:44 46 (1982)); canine kidney cells (MDCK) (ATCC CCL 34 and CRL 6253), HEK 293 (ATCC CRL 1573), WI-38 cells (ATCC CCL 75) (ATCC: American Type Culture Collection, Rockville, Md.), MCF-7 cells, MDA-MB-438 cells, U87 cells, A127 cells, HL60 cells, A549 cells, SP10 cells, DOX cells, SHSY5Y cells, Jurkat cells, BCP-1 cells, GH3 cells, 9L cells, MC3T3 cells, C3H-10T1/2 cells, NIH-3T3 cells, C6/36 cells human lymphoblast cells (e.g. GEX) and PER.C6® cells. The use of mammalian tissue cell culture to express polypeptides is discussed generally in Winnacker, FROM GENES TO CLONES (VCH Publishers, N.Y., N.Y., 1987).

Exemplary plant cells include, for example, Arabidopsis thaliana, rape seed, corn, wheat, rice, tobacco etc.) (Staub, et al. 2000 Nature Biotechnology 1(3): 333-338 and McGarvey, P. B., et al. 1995 Bio-Technology 13(13): 1484-1487; Bardor, M., et al. 1999 Trends in Plant Science 4(9): 376-380). Exemplary insect cells (for example, Spodoptera frugiperda Sf9, Sf21, Trichoplusia ni, etc. Exemplary bacteria cells include Escherichia coli. Various yeasts and fungi such as Pichiapastoris, Pichia methanolica, Hansenula polymorpha, and Saccharomyces cerevisiae can also be selected.

Culture Media and Processing

The methods described herein can include determining and/or selecting media components or culture conditions which result in the production of a desired glycan property or properties. Culture parameters that can be determined include media components, pH, feeding conditions, osmolarity, carbon dioxide levels, agitation rate, temperature, cell density, seeding density, timing and sparge rate.

Changes in production parameters such the speed of agitation of a cell culture, the temperature at which cells are cultures, the components in the culture medium, the times at which cultures are started and stopped, variation in the timing of nutrient supply can result in variation of a glycan properties of the produced glycoprotein product. Thus, methods described herein can include one or more of: increasing or decreasing the speed at which cells are agitated, increasing or decreasing the temperature at which cells are cultures, adding or removing media components, and altering the times at which cultures are started and/or stopped.

Sequentially selecting a production parameters or a combination thereof, as used herein, means a first parameter (or combination) is selected, and then a second parameter (or combination) is selected, e.g., based on a constraint imposed by the choice of the first production parameter.

Media

The methods described herein can include determining and/or selecting a media component and/or the concentration of a media component that has a positive correlation to a desired glycan property or properties. A media component can be added in or administered over the course of glycoprotein production or when there is a change in media, depending on culture conditions. Media components include components added directly to culture as well as components that are a byproduct of cell culture.

Media components include, e.g., buffer, amino acid content, vitamin content, salt content, mineral content, serum content, carbon source content, lipid content, nucleic acid content, hormone content, trace element content, ammonia content, co-factor content, indicator content, small molecule content, hydrolysate content and enzyme modulator content.

Table 3 provides examples of various media components that can be selected.

TABLE 3

| | |
|---|---|
| amino acids | sugar precursors |
| Vitamins | Indicators |
| Carbon source (natural and unnatural) | Nucleosides or nucleotides |
| Salts | butyrate or organics |
| Sugars | DMSO |
| Sera | Animal derived products |
| Plant derived hydrolysates | Gene inducers |
| sodium pyruvate | Non natural sugars |
| Surfactants | Regulators of intracellular pH |
| Ammonia | Betaine or osmoprotectant |
| Lipids | Trace elements |
| Hormones or growth factors | minerals |
| Buffers | Non natural amino acids |
| Non natural amino acids | Non natural vitamins |

Exemplary buffers include Tris, Tricine, HEPES, MOPS, PIPES, TAPS, bicine, BES, TES, cacodylate, MES, acetate, MKP, ADA, ACES, glycinamide and acetamidoglycine.

The media can be serum free or can include animal derived products such as, e.g., fetal bovine serum (FBS), fetal calf serum (FCS), horse serum (HS), human serum, animal derived serum substitutes (e.g., Ultroser G, SF and HY; non-fat dry milk; Bovine EX-CYTE), fetuin, bovine serum albumin (BSA), serum albumin, and transferrin. When serum free media is selected lipids such as, e.g., palmitic acid and/or steric acid, can be included.

Lipids components include oils, saturated fatty acids, unsaturated fatty acids, glycerides, steroids, phospholipids, sphingolipids and lipoproteins.

Exemplary amino acid that can be included or eliminated from the media include alanine, arginine, asparagine, aspartic acid, cysteine, glutamic acid, glutamine, glycine, histidine, proline, isoleucine, leucine, lysine, methionine, phenylalanine, proline, serine, threonine, tryptophan, tyrosine and valine.

Examples of vitamins that can be present in the media or eliminated from the media include vitamin A (retinoid), vitamin B1 (thiamine), vitamin B2 (riboflavin), vitamin B3 (niacin), vitamin B5 (pantothenic acid), vitamin B6 (pyroxidone), vitamin B7 (biotin), vitamin B9 (folic acid), vitamin B12 (cyanocobalamin), vitamin C (ascorbic acid), vitamin D, vitamin E, and vitamin K.

Minerals that can be present in the media or eliminated from the media include bismuth, boron, calcium, chlorine, chromium, cobalt, copper, fluorine, iodine, iron, magnesium, manganese, molybdenum, nickel, phosphorus, potassium, rubidium, selenium, silicon, sodium, strontium, sulfur, tellurium, titanium, tungsten, vanadium, and zinc. Exemplary salts and minerals include CaCl₂ (anhydrous), CuSO₄5H₂O, Fe(NO₃).9H₂O, KCl, KNO₃, KH₂PO₄, MgSO₄ (anhydrous), NaCl, NaH₂PO₄H₂O, NaHCO₃, Na₂SeO₃ (anhydrous), ZnSO₄.7H₂O; linoleic acid, lipoic acid, D-glucose, hypoxanthine 2Na, phenol red, putrescine 2HCl, sodium pyruvate, thymidine, pyruvic acid, sodium succinate, succinic acid, succinic acid.Na.hexahydrate, glutathione (reduced), para-aminobenzoic acid (PABA), methyl linoleate, bacto peptone G, adenosine, cytidine, guanosine, 2′-deoxyadenosine HCl, 2′-deoxycytidine HCl, 2′-deoxyguanosine and uridine. When the desired glycan characteristic is decreased fucosylation, the production parameters can include culturing a cell, e.g., CHO cell, e.g., dhfr deficient CHO cell, in the presence of manganese, e.g., manganese present at a concentration of about 0.1 μM to 50 μM. Decreased fucosylation can also be obtained, e.g., by culturing a cell (e.g., a CHO cell, e.g., a dhfr deficient CHO cell) at an osmolality of about 350 to 500 mOsm. Osmolality can be adjusted by adding salt to the media or having salt be produced as a byproduct as evaporation occurs during production.

Hormones include, for example, somatostatin, growth hormone-releasing factor (GRF), insulin, prolactin, human growth hormone (hGH), somatotropin, estradiol, and progesterone. Growth factors include, for example, bone morphogenic protein (BMP), epidermal growth factor (EGF), basic fibroblast growth factor (bFGF), nerve growth factor (NGF), bone derived growth factor (BDGF), transforming growth factor-beta1 (TGF-beta1), [Growth factors from U.S. Pat. No. 6,838,284 B2], hemin and NAD.

Examples of surfactants that can be present or eliminated from the media include Tween-80 and pluronic F-68.

Small molecules can include, e.g., butyrate, ammonia, non natural sugars, non natural amino acids, chloroquine, and betaine.

In some embodiments, ammonia content can be selected as a production parameter to produce a desired glycan characteristic or characteristics. For example, ammonia can be present in the media in a range from 0.001 to 50 mM. Ammonia can be directly added to the culture and/or can be produced as a by product of glutamine or glucosamine. When the desired glycan characteristic is one or more of an increased number of high mannose structures, decreased fucosylation and decreased galactosylation, the production parameters selected can include culturing a cell (e.g., a CHO cell, e.g., a dhfr deficient CHO cell) in the presence of ammonia, e.g., ammonia present at a concentration of about 0.01 to 50 mM. For example, if the desired glycan characteristic includes decreased galactosylation, production parameters selected can include culturing a cell (e.g., a CHO cell, e.g., a dhfr deficient CHO cell) in serum containing media and in the presence of ammonia, e.g., ammonia present at a concentration of about 0.01 to 50 mM.

Another production parameter is butyrate content. The presence of butyrate in culture media can result in increased galactose levels in the resulting glycoprotein preparation. Butyrate provides increased sialic acid content in the resulting glycoprotein preparation. Therefore, when increased galactosylation and/or sialylation is desired, the cell used to produce the glycoprotein (e.g., a CHO cell, e.g., a dhfr deficient CHO cell) can be cultured in the presence of butyrate. In some embodiments, butyrate can be present at a concentration of about 0.001 to 10 mM, e.g., about 2 mM to 10 mM. For example, if the desired glycan characteristic includes increased sialylation, production parameters selected can include culturing a cell (e.g., a CHO cell, e.g., a dhfr deficient CHO cell) in serum containing media and in the presence of butyrate, e.g., butyrate present at a concentration of about 2.0 to 10 mM. Such methods can further include selecting one or more of adherent culture conditions and culture in a T flask.

Physiochemical Parameters

Methods described herein can include selecting culture conditions that are correlated with a desired glycan property or properties. Such conditions can include temperature, pH, osmolality, shear force or agitation rate, oxidation, spurge rate, growth vessel, tangential flow, DO, CO₂, nitrogen, fed batch, redox, cell density and feed strategy. Examples of physiochemical parameters that can be selected are provided in Table 4.

TABLE 4

| | |
|---|---|
| Temperature | DO |
| pH | CO₂ |
| osmolality | Nitrogen |
| shear force, or agitation rate | Fed batch |
| oxidation | Redox |
| Spurge rate | Cell density |
| Growth vessel | Perfusion culture |
| Tangential flow | Feed strategy |
| Batch | |

For example, the production parameter can be culturing a cell under acidic, neutral or basic pH conditions. Temperatures can be selected from 10 to 42° C. For example, a temperature of about 28 to 36° C. does not significantly alter galactosylation, fucosylation, high mannose production, hybrid production or sialylation of glycoproteins produced by a cell (e.g., a CHO cell, e.g., a dhfr deficient CHO cell) cultured at these temperatures. In addition, any method that slows down the growth rate of a cell may also have this effect. Thus, temperatures in this range or methods that slow down growth rate can be selected when it is desirable not to have this parameter of production altering glycosynthesis.

In other embodiments, carbon dioxide levels can be selected which results in a desired glycan characteristic or characteristics. CO₂ levels can be, e.g., about 5%, 6%, 7%, 8%, 9%, 10%, 11%, 13%, 15%, 17%, 20%, 23% and 25% (and ranges in between). In one embodiment, when decreased fucosylation is desired, the cell can be cultured at CO₂ levels of about 11 to 25%, e.g., about 15%. CO₂ levels can be adjusted manually or can be a cell byproduct.

A wide array of flasks, bottles, reactors, and controllers allow the production and scale up of cell culture systems. The system can be chosen based, at least in part, upon its correlation with a desired glycan property or properties.

Cells can be grown, for example, as batch, fed-batch, perfusion, or continuous cultures.

Production parameters that can be selected include, e.g., addition or removal of media including when (early, middle or late during culture time) and how often media is harvested; increasing or decreasing speed at which cell cultures are agitated; increasing or decreasing temperature at which cells are cultured; adding or removing media such that culture density is adjusted; selecting a time at which cell cultures are started or stopped; and selecting a time at which cell culture parameters are changed. Such parameters can be selected for any of the batch, fed-batch, perfusion and continuous culture conditions.

### Other Embodiments

Also disclosed herein are methods of making, or providing, a glycoprotein having a target glycan structure, e.g., by inhibiting or promoting the addition of a monosaccharide moiety to a protein or glycoprotein. The method includes providing a transgenic animal or plant having or subject to a manipulation that increases or decreases the level of the activity of a nucleoside diphosphatase, e.g., the level of activity in the Golgi, and which manipulation thereby promotes the formation of said target glycan structure; maintaining the transgenic animal or transgenic plant under conditions that allow for production of the glycoprotein; and optionally, separating the glycoprotein or protein having a target glycan structure from at least one component with which the glycoprotein was produced, thereby providing a glycoprotein having a target glycan structure. The method can include any of the manipulations described herein that are appropriate for transgenic animal or transgenic plant production. The method may be used to make target glycan structures described herein.

Also disclosed herein are methods of monitoring a process. The method includes, optionally, selecting a target glycan structure; optionally, selecting a transgenic animal or transgenic plant on the basis of it having or subject to a manipulation that increases or decreases the level of the activity of a nucleoside diphosphatase, e.g., the level of activity in the Golgi, and which manipulation thereby promotes the formation of said target glycan structure; providing a transgenic animal or transgenic plant having or subject to a manipulation that increases or decreases the level of the activity of a nucleoside diphosphatase, e.g., the level of activity in the Golgi; maintaining the transgenic animal or transgenic plant under conditions that allow for production of the glycoprotein; and evaluating (directly or indirectly) a glycan produced by the transgenic animal or transgenic plant, to thereby monitor the process.

### EXAMPLES

### Example 1

Chinese hamster ovary (CHO) cells were supplemented with the nucleoside uridine. Cells were harvested, and snap frozen, while culture supernatant was harvested and CTLA4Ig harvested by protein A purification for subsequent analysis. Cells were then subjected to nucleotide sugar extraction using standard methods. In short, this was performed with chloroform:methanol:water (2:4:1), the pellets discarded, and the resulting extraction dried down. The dried material was subsequently resuspended in 500 ul of 10% butanol in water and then extracted with 1 ml of 90% butanol in water. The butanol phase was discarded and the acqueous subjected to a second butanol extraction. The final aqueous phase was dried down and the sugar nucleotides further isolated by PGC chromatography eluting off with 25% acetonitrile (v/v) containing 50 mM triethylammonium acetate. For quantification, sugar-nucletides were resolved with RP chromatography.

Protein product was deglycosylated with PNGase F for 18 hours and the resulting glycans isolated with PGC chromatography. The glycans were subsequently labeled with either ¹²C Aniline (control conditions) or ¹³C Aniline (cultures supplemented with uridine) by reductive amination. The ¹²C and ¹³C labeled glycans were then mixed in equal ratios and subjected to LC-MS analysis. Resulting MS peak areas were quantified and for each glycan the ratio of the control vs. the uridine treated samples was determined.

Methods of the invention rely on the inhibition of nucleoside diphosphatase to increase NDP levels, e.g., UDP levels, to thereby inhibit glycosyltransferase and reduce glycosylation. This example gives proof-of-principle by using the addition of uridine to conveniently model an increase in UDP (and the resulting inhibition of glycosylation) which results from a manipulation of the invention, e.g., inhibition of uridine diphosphatase. As shown in the example, an increase in UDP results in a reduction of gylcosylation.

In this example, the levels of the uridine sugars increased significantly (Table 5) including UDP-Gal and UDP-GlcNAc. The level of the monogalactosylated species on the protein product, rises as compared to the untreated control illustrating incomplete galactosylation. This is consistent with results of a mathematical model of cellular glycosylation which shows that, without alteration of the activity or concentration of the phosphatase, the level of uridine diphosphate rises to such a level that it becomes inhibitory to the activity of the galactosyltransferase. Taken together these data indicate the decrease in galactosylation observed in the experimental system is likely due to the feedback inhibition of the UDP sugar.

TABLE 5
Intracellular sugar-nucleotide levels from CHO cells cultured
with Uridine supplementation. Data are expressed as the
average increase in sugar-nucloetide above no
supplemented cells +/− the SD of duplicate determinants

| | CMP- | UDP- | UDP- | UDP- | GDP- | GDP- |
|---|---|---|---|---|---|---|
| | NeuAc | Gal | Glc | GlcNAc | Man | Fuc |
| Uridine 2 x | 32.13 | 168.97 | 409.16 | 402.7 | 6.06 | 6.5 |
| Uridine 10 x | 50.52 | 389.06 | 1261.96 | 2280.49 | 12.45 | 8.98 |

1. A method of inhibiting or promoting the addition of a galactosyl moiety from a UDP-galactose glycosyl donor to an acceptor glycoprotein or protein, comprising:
providing a cell having or subject to a manipulation that decreases the level of the activity of a UDP nucleoside diphosphatase,
and increases the proportion of mono-galactosylated glycans;
optionally, separating said glycoprotein or protein having a target glycan structure resulting from said inhibiting or promoting from at least one component with which said cell or batch of cells was cultured; and
optionally, analyzing said glycoprotein to confirm the presence of the target glycan structure,
thereby inhibiting or promoting the addition of a glycan galactosyl moiety to an acceptor glycoprotein or protein.

2. The method of claim 1, further comprising one or more of:
optionally, selecting a target glycan structure;
optionally, selecting a cell on the basis of the cell having or subject to a manipulation that increases or decreases the level of the activity of a UDP nucleoside diphosphatase; and
optionally, analyzing said glycoprotein to confirm the presence of the target glycan structure.

3. The method of claim 1, further comprising evaluating a glycan on the surface of said cell or batch of cultured cells in order to determine if said target glycan structure is present on a glycoprotein produced by said cell or batch of cultured cells.

4. The method of claim 3, wherein said evaluation comprises evaluating a glycan on the surface of said cell or batch of cultured cells, to determine a property of said glycan, comparing the property to a reference, to thereby determine if said target glycan structure is present on the product.

5.-7. (canceled)

8. The method of claim 1, wherein the amount (or proportion) of mono-galactosylated glycans is increased relative to the amount (or proportion) of mono-galactosylated glycans in a preparation of glycoproteins made by a cell or batch of cultured cells not subjected to the manipulation (in other words, a ratio of proportions).

9.-80. (canceled)

81. The method of claim 1, wherein the glycoprotein is an N-linked glycoprotein, an O-linked glycoprotein, a cell surface receptor, an immunoglobulin super family or portion thereof, a hormone or is selected from Table 1.

82.-84. (canceled)

85. The method of claim 1, further comprising isolating the glycoprotein from the cell or batch of cultured cells:
combining the glycoprotein having a target glycan structure with a pharmaceutically acceptable component;
evaluating (directly or indirectly) the glycan structure of the glycoprotein;
analyzing the glycoprotein to determine if the target glycan structure is present.

86.-87. (canceled)

88. The method of claim 85, wherein evaluating comprises
evaluating the level of the UDP nucleoside diphosphate and
determining a value for a property of the glycan structure on the glycoprotein and comparing that value with a reference value.

89.-90. (canceled)

91. The method of claim 85, wherein the glycoprotein is analyzed by a method selected from the group consisting of: chromatographic methods, mass spectrometry (MS) methods, electrophoretic methods, nuclear magnetic resonance (NMR) methods, monosaccharide analysis, fluorescence methods, UV-VIS absorbance, enzymatic methods, use of a detection molecule, and combinations thereof.

92. The method of claim 1, comprising selecting one or both of a target glycan structure or a glycoprotein sequence for use in the method.

93. The method of claim 1, wherein the culture is supplemented with a nucleoside; or
cobalt, sodium butyrate, glucosamine, ammonia, fucose, manganese, mannose or a monosaccharide.

94.-95. (canceled)

96. The method of claim 1, where the manipulation is, or is a product of,
a genetic manipulation which decreases the level of a UDP nucleoside diphosphatase activity; or
a selection for a decreased UDP nucleoside diphosphatase activity or decreased level of UDP nucleoside diphosphatase activity,
a selection for the production of a target glycan structure, a decreased glycosylation or a target decreased level of glycosylation;
comprises contact with or inclusion in or on the cell or batch of cultured cells or an exogenous inhibitor of a UDP nucleoside diphosphatase;
comprises contact with or inclusion in or on the cell or batch of cultured cells of a sequence specific nucleic acid-based inhibitor of the gene that encodes a UDP nucleoside diphosphatase; or
comprises contact with or inclusion in or on the cell or batch of cultured cells of an inhibitor of a UDP nucleoside diphosphatase activity.

97.-108. (canceled)

109. The method of claim 1, wherein one or more of said cell or said batch of cultured cells, said manipulation, and said glycoprotein, is selected on the basis that it or the combination will provide a glycoprotein having the target glycan structure;
the target glycan structure is increased, remains the same, or is decreased, as compared to what would be seen in the absence of the manipulation; or
a component of the target glycan structure is transferred by a glycosyltransferase from a glycosyl donor to a protein acceptor or glycoprotein acceptor to provide said glycoprotein and a nucleoside diphosphate, and the glycosyl donor is UDP-galactose, UDP-N-acetylglucosamine, UDP-N-acetylgalactosamine, GDP-fucose or GDP-mannose.

110.-111. (canceled)

112. A method of making, or providing, a glycoprotein having a target glycan structure, e.g., by inhibiting or promoting the addition of a monosaccharide moiety to a protein or glycoprotein, comprising:
providing a cell having or subject to a manipulation that increases or decreases the level of the activity of a nucleoside diphosphatase, e.g., the level of activity in the Golgi, and which manipulation thereby promotes the formation of said target glycan structure;
culturing said cell, e.g., to provide a batch of cultured cells;
optionally, separating the glycoprotein or protein having a target glycan structure from at least one component with which said cell or batch of cells was cultured;
optionally, analyzing said glycoprotein to confirm the presence of the target glycan structure; and
optionally, comparing the structure of said target glycan structure present on a glycoprotein from said cultured cell or batch of cells to a reference, and determining if said target glycan structure present on a glycoprotein from said cultured cell or batch of cells differs from the corresponding glycan structure formed by a cell that lacks the manipulation
thereby providing a glycoprotein having a target glycan structure.

113.-222. (canceled)

223. A method of monitoring a process, e.g., a process of culturing cells, e.g., cells of a selected type, to produce a product, comprising:
optionally, selecting a target glycan structure;
optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure;
providing a cell having or subject to a manipulation that increases or decreases the level of the activity of a nucleoside diphosphatase, e.g., the level of activity in the Golgi;
culturing said cell, e.g., to provide a batch of cultured cells; and
evaluating (directly or indirectly) a glycan complement, glycan component or glycan structure produced by the cell or the batch of cultured cells, to thereby monitor the process.

224.-238. (canceled)

239. A method of controlling a process for making a glycoprotein having a target glycan structure, comprising:
(1) providing a glycoprotein made by the process of:
optionally, selecting a target glycan structure;
optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure;
providing a cell having or subject to a manipulation that increases or decreases the level of the activity of a nucleoside diphosphatase, e.g., the level of activity in the Golgi; and
culturing the cell to provide a glycoprotein and, e.g., to form a batch of cultured cells;
(2) evaluating (directly or indirectly) the glycan structure of the glycoprotein,
(3) responsive to said evaluation, selecting a production parameter, e.g., a culture condition, e.g., a level of a nutrient or other component in the culture medium,
to thereby control the process for making a glycoprotein having a target glycan structure.

240.-242. (canceled)

243. A method of making a glycoprotein, comprising:
(a) providing, acknowledging, selecting, accepting, or memorializing a defined, desired or preselected target glycan structure for the glycoprotein,
(b) optionally providing a cell manipulated to increase or decrease the level of a nucleoside diphosphatase activity,
(c) culturing a cell manipulated to increase or decrease the level of a nucleoside diphosphatase activity, e.g., to form a batch of cultured cells, and
(d) isolating from the cell or batch of cultured cells a glycoprotein having the desired target glycan structure,
thereby making a glycoprotein.

244. A method of making a glycoprotein, comprising:
(a) providing, acknowledging, selecting, accepting, or memorializing a defined, desired or preselected target glycan structure for the glycoprotein, chosen, e.g., from Table 1;
(b) optionally, providing, acknowledging, selecting, accepting, or memorializing a manipulation from Table 2;
(c) culturing a cell having the manipulation, e.g., to form a batch of cultured cells;
(d) isolating from the cell or batch of cultured cells a glycoprotein having the desired target glycan structure,
thereby making a glycoprotein.

245. A method of formulating a pharmaceutical composition comprising:
contacting a glycoprotein made by a method described herein with a pharmaceutically acceptable substance, e.g., an excipient or diluent.

246. A reaction mixture comprising a manipulated cell described herein and a culture medium, optionally including secreted glycoprotein having a target glycan structure.

247. A pharmaceutical preparation of a glycoprotein described herein or made by a method described herein, wherein the glycoprotein is selected from Table 1.

248. A glycoprotein selected from Table 1 having a target glycan structure selected from Table 2.

249. A method of making, or providing, a glycoprotein having a target glycan structure, e.g., by inhibiting or promoting the addition of a monosaccharide moiety to a protein or glycoprotein, comprising:
optionally, selecting a target glycan structure;
selecting a cell, preferably on the basis that it produces a protein having the primary amino acid sequence of said glycoprotein but which protein when provided by said cell lacks said target glycan structure;
optionally, selecting a manipulation, e.g., selecting the manipulation on the basis that the manipulation increases or decreases the level of the activity of a nucleoside diphosphatase, e.g., the level of activity in the Golgi, and which manipulation thereby promotes the formation of said target glycan structure
providing said manipulation to said cell to provide a cell having or subject to a manipulation that increases or decreases the level of the activity of a nucleoside diphosphatase, e.g., the level of activity in the Golgi, and which manipulation thereby promotes the formation of said target glycan structure;
culturing said selected cell, e.g., to provide a batch of cultured cells;
optionally, separating the glycoprotein having a target glycan structure from at least one component with which the cell or said batch of cultured cells was cultured;
optionally, analyzing said glycoprotein to confirm the presence of the target glycan structure
thereby making, or providing, a glycoprotein having a target glycan structure, e.g., by inhibiting or promoting the addition of a monosaccharide moiety to a protein or glycoprotein.

250. A method of providing a cell that makes a glycoprotein having a target glycan structure, comprising:
optionally, selecting a target glycan structure;
selecting a cell, preferably on the basis that it produces a protein having the primary amino acid sequence of said glycoprotein but which protein lacks said target glycan structure;
optionally, selecting a manipulation, e.g., selecting the manipulation on the basis that the manipulation increases or decreases the level of the activity of a nucleoside diphosphatase, e.g., the level of activity in the Golgi, and which manipulation thereby promotes the formation of said target glycan structure;
providing said manipulation to said cell to provide a cell having or subject to a manipulation that increases or decreases the level of the activity of a nucleoside diphosphatase, e.g., the level of activity in the Golgi, and which manipulation thereby promotes the formation of said target glycan structure;
optionally producing glycoprotein from said cell and determining if said glycoprotein has said target glycan structure,
thereby providing a cell that makes a glycoprotein having a target glycan structure.

251. A method of selecting a cell suitable for the production of protein having a target gylcan, comprising:
optionally, selecting a target glycan structure, e.g., from a list comprising a plurality of target glycan structures (in embodiments the list is also provided), and optionally memorializing said selected target glycan structure;
optionally, selecting a cell on the basis of the cell having or subject to a manipulation that increases or decreases the level of the activity of a nucleoside diphosphatase, e.g., the level of activity in the Golgi, and which manipulation thereby promotes the formation of said target glycan structure (in embodiments the manipulation is from a list comprising a plurality of manipulations, and in embodiments the list is also provided);
providing a cell having or subject to a manipulation that increases or decreases the level of the activity of a nucleoside diphosphatase, e.g., the level of activity in the Golgi;
culturing said cell to provide a plurality of progeny cells; and
selecting one of said progeny cells.

252. (canceled)
